(19) United States
(12) Patent Application Publication
(10) Pub. No.: US 2025/0127784 A1
(43) Pub. Date: Apr. 24, 2025

(54) Treatment of Telangiectasia

(71) Applicant: IMPERIAL COLLEGE INNOVATIONS LIMITED, South Kensington, London (GB)

(72) Inventor: Claire SHOVLIN, London (GB)

(21) Appl. No.: 18/836,510

(22) PCT Filed: Feb. 9, 2023
(86) PCT No.: PCT/GB2023/050295

§ 371 Date: Aug. 7, 2024

(30) Foreign Application Priority Data
Feb. 9, 2022 (GB) 2201683.6

(51) Int. Cl. A61K 31/519; A61P 7/04; C12Q 1/6883

(52) U.S. Cl. CPC A61K 31/519; A61P 7/04; C12Q 1/6883; C12Q 2600/106

(57) ABSTRACT

The present invention relates to the treatment of telangiectasia, particularly telangiectasia associated with hereditary haemorrhagic telangiectasia (HHT). The present invention provides treatments for telangiectasia associated with HHT, and HHT-induced anaemia and/or haemorrhage. Methods of identifying patients suitable for such treatment are also provided.

### FIELD OF THE INVENTION

The present invention relates to the treatment of telangiectasia, particularly telangiectasia associated with hereditary haemorrhagic telangiectasia (HHT). The present invention provides treatments for telangiectasia associated with HHT, and HHT-induced anaemia and/or haemorrhage. Methods of identifying patients suitable for such treatment are also provided.

### BACKGROUND OF THE INVENTION

Hereditary haemorrhagic telangiectasia (HHT) is an autosomal dominant blood vessel disorder, resulting from a single DNA variant (loss-of-function mutation) in one of four genes encoding activin receptor like kinase 1 (ACVRL1), endoglin (ENG), Mothers Against Decapentaplegic Homolog 4 (SMAD4) or bone morphogenetic protein 9 (BMP9; (aka growth and differentiation factor 2, (GDF2). HHT affects 1 in 5000 individuals. The disease is characterised by the abnormal formation of blood vessels, where a capillary bed is lacking between arteries and veins, resulting in two types of vascular abnormalities: telangiectasia and arteriovenous malformations (AVMs).

Severe manifestations of HHT often involve large AVMs, particularly pulmonary, hepatic and cerebral AVMs. AVMs can result in many complications, for example approximately 2-3% of HHT patients will require liver transplantation or antiangiogenic therapies due to hepatic AVMs that are causing high output cardiac failure. Therefore, to date, most research has focused on targeting these large AVMs, with the intention of identifying potential treatments for these more severe manifestations.

However, telangiectasia and associated bleeding and anaemia are even more common in HHT patients. High pressure arterial blood flowing directly into thin-walled veins can result in rupture of the abnormal telangiectatic vessels, leading to haemorrhage. Symptoms can include nosebleeds, iron deficiency and anaemia, gastrointestinal bleeding, and in some cases, high output cardiac failure. The treatment options available depend on the severity of a patient's bleeding.

It is HHT bleeding, rather than large AVMs, that has a particularly significant impact on patients' quality of life. Most HHT patients experience nose bleeds sufficient to cause iron deficiency anaemia. Whilst many of these patients can be treated with oral iron supplements, some experience severe anaemia, requiring treatments such as weekly blood transfusions, iron infusions, intravenous Bevacizumab (Avastin), or even surgery. Invasive treatments such as regular blood transfusions and i.v. medication typically require regular hospital or clinic visits by a patient, and require administration by medically trained staff, increasing costs and the time required for treatment. Patient compliance can also be an issue when regular hospital visits are required. There is therefore a need for effective, non-invasive treatments for telangiectasia and HHT.

The present invention overcomes one or more of the above-mentioned problems. In particular, it is an object of the present invention to provide treatments for telangiectasia, particularly telangiectasia associated with HHT, that are less invasive than conventional treatments. These treatments can improve patient compliance and quality of life.

### SUMMARY OF THE INVENTION

The present inventors are the first to elucidate a role for ERK1/2 signalling in the development of telangiectasia in HHT. In particular, the present inventors are the first to appreciate that an increase in ERK1/2 signalling following stress-induced up-regulation of MEKK1 leads to the development of telangiectasia in HHT. The inventors have shown that treatment of ENG^{+/-} HHT patient with the MEK1/2 inhibitor trametinib surprisingly treats telangiectasia, and particularly anaemia and haemorrhage associated with telangiectasia in HHT, but has no effect on the patient's AVMs. Therefore, the present inventors are the first to provide a method of treating telangiectasia in HHT, and telangiectasia-associated haemorrhage and anaemia using a MEK1/2 inhibitor. This represents a step-change from conventional HHT treatments, which either focus on treating the larger AVMs, or use invasive treatments (such as blood transfusions) to treat telangiectasia-associated aspects of the disease. The present invention therefore offers a less-invasive treatment which is particularly suited to treating both severe and less severe cases of HHT, with the potential to reduce the need for hospital/clinic visits, reduce care costs, increase patient compliance, and increase patient quality of life.

Accordingly, the invention provides a MEK1/2 inhibitor for use in the treatment of telangiectasia associated with hereditary haemorrhagic telangiectasia (HHT). The telangiectasia may be nasal telangiectasia. Said inhibitor may treat or prevent HHT induced anaemia and/or haemorrhage. The HHT induced anaemia may be associated with recurrent haemorrhages and/or the HHT induced haemorrhage may be recurrent. The anaemia and/or haemorrhage to be treated may not be responsive to dietary iron supplements. The anaemia and/or haemorrhage may otherwise require treatment comprising surgery, an anti-angiogenic agent and/or blood transfusion. Said inhibitor may be trametinib. The dose of the MEK1/2 inhibitor may be less than 2 mg/day. The MEK1/2 inhibitor may be for oral, intravenous or subcutaneous administration. In some preferred embodiments, said inhibitor is trametinib for oral administration. The MEK1/2 inhibitor may be administered at intervals daily, weekly, fortnightly, or monthly, preferably daily.

The invention further provides the use of a MEK1/2 inhibitor in the manufacture of a medicament for the treatment of telangiectasia associated with HHT. Said inhibitor may treat or prevent HHT induced anaemia and/or haemorrhage. Said inhibitor may be trametinib.

The invention also provides a method for treating telangiectasia associated with HHT, the method comprising administrating a therapeutically effective amount of a MEK1/2 inhibitor to a patient in need thereof. Said inhibitor may treat or prevent HHT induced anaemia and/or haemorrhage. Said inhibitor may be trametinib.

The invention further provides a method of identifying a patient as suitable for use of a MEK1/2 inhibitor for treating or preventing telangiectasia associated with HHT according to the invention, said method comprising determining and/or quantifying one or more of a patient's: (a) red cell indices; (b) impaired activity; (c) lack responsiveness to dietary or oral iron supplements; and/or (d) lack of tolerance to bleeding and/or anaemia.

### BRIEF DESCRIPTION OF THE DRAWINGS

FIG. 1: A Signalling pathways activated by non-HHT vascular malformation syndrome gene mutations that may be inherited, or of acquired, sporadic aetiology in somatic tissues. Normal activators are shown by thin arrows, normal inhibitors by thin lines and bars, pathway names by grey italics, receptors are flanked in bold at membranes, and other moieties named (in dotted circles/ovals) or unnamed (empty dotted circle). Vascular malformation genes are listed outside of the main box: thick bold boxes and barred lines indicate pathway inhibitors where inactivating mutations lead to pathway activation (e.g. loss of function mutations in PTEN, EPHB4 and RASA1). Thick dotted boxes and arrows indicate where pathway activation is caused by rare activating mutations in [from bottom left: AKT, GNAQ and GNA11 encoding G protein subunits, PIK3CA, MAP3K3, TIE2 (TEK), KRAS, NRAS, BRAF and MAP2K1). Highlighted specifically are VEGF and trametinib inhibiting the Ras-Raf-MEK-ERK cascade. None of the ACVRL1, ENG, SMAD4 or GDF2 protein products (ALK1, ENG, SMAD4, BMP9) or TGF-BMP signalling pathways appear on the map. Base signalling map adapted from open sources including Kanehisa Laboratories (Protein Sci (2019) 28:1947-1951, with particular modifications as listed above. B Signalling pathways disrupted by HHT gene mutations in genes encoding proteins that transmit extracellular BMP/TGF-beta superfamily ligands through serine threonine kinase signalling pathways. HHT is usually caused by heterozygous loss of function mutations in ACVRL1, ENG or SMAD4, with BMP9 newly implicated in HHT.

FIG. 2: The integrated stress response (ISR): diverse cytosolic stresses are sensed by four kinases, protein kinase RNA-like ER kinase (PERK), heme-regulated elF2a (HRI) kinase, general control nonderepressible 2 (GCN2) kinase and protein kinase R (PKR). PERK, HRI, GCN2 and PKR can each phosphorylate the a subunit of eukaryotic initiation factor 2 (elF2a) which inhibits assembly of the charged methionyl-initiator tRNA elF2.GTP.methionyl-intiator tRNA ternary complex (TC) required for new protein synthesis. This inhibits translation-dependent nonsense mediating decay (NMD) which allows PTC-harbouring transcripts to persist, and increases full length protein translation by ribosomal reinitiation for genes regulated by upstream open reading frames. These include ATF4 encoding activating transcription factor 4 that acts as a master transcription factor during the ISR. Stress conditions modify export factors preventing bulk mRNA export via nuclear RNA export factor 1 protein (pNXF1), and dissociating other RNA binding proteins such as the RNA binding motif protein 45 (pRBM45), so they form bulk mRNAs for nuclear transport, and aggregate in nuclear foci. Where the ISR is not transient and stress sustained, apoptosis results.

FIG. 3: Nosebleed frequency, iron use, and blood transfusion use indices compared to HHT population of N=202 (as described in Shovlin et al. (Blood Rev. 2010; 24 (6): 203-19)) Details of bleeding, iron and transfusion are provided before (“Pre”) and after (“Post”) 10 months of Trametinib. Base graphs are black and white versions of colour graphs originally published by Shovlin in 2010 to categorise nosebleed frequency, use of oral iron and use of blood transfusions across 202 consecutive patients with hereditary haemorrhagic telangiectasia (HHT) and pulmonary AVMs that she had reviewed between 1999-2005 under LREC 2000/5764 and LREC 00/5792. A Maximum nose bleed frequency, plotted for patient, super-imposed on base graph published on 202 HHT patients. Pre Trametinib, the nosebleed frequency was in the highest category as defined in 2010. After 10 months of Trametinib, the nosebleed frequency was in the third lowest category as defined in 2010. B Oral iron and blood transfusion use by category of nose bleed frequency, plotted for patient superimposed on base graph published on the same 202 HHT patients. C Maximum nose bleed frequency by age quartile (Qu), plotted for patient, superimposed on base graph published on 202 HHT patients. This graph plots patients experiencing nosebleeds in the two most severe categories (daily or weekly). Note that after 10 months of Trametinib treatment, the patient no longer fitted into either of these categories.

FIG. 4: Nosebleed severity quantified by haemorrhage adjusted iron requirements (HAIR) compared to 2013 population of N=50 (as described in Finnamore et al. (PLOS One 2013; 8 (10): e76516)). Details of the haemorrhage adjusted iron requirement (HAIR) response to 10 months of Trametinib. Base Graphs are black and white versions of colour graphs originally published in 2013 to define the true daily iron requirement of patients based on the recommended daily intake (which is higher in menstruating women) plus an adjustment to reflect nosebleed severity, calculated across the 50 patients with hereditary haemorrhagic telangiectasia (HHT) recruited to NRES 11/H0803/8. A Haemorrhage adjusted iron requirements (HAIR) plotted for 50 participants in rank order on the x axis, with the patient's values superimposed. HAIR (in mg of iron per day) was calculated by the recommended daily iron intake plus the iron required to replace losses calculated using the nosebleed severity index of nosebleed frequency x nosebleed duration x nosebleed intensity, as detailed in Finnamore et al 2013. Pre Trametinib, HAIR was in the 4th highest grouping as defined in 2013. After 10 months HAIR was in the lowest grouping as defined in 2013. B Haemorrhage adjusted iron requirement (HAIR) plotted for 50 participants based on their menstrual status (left) and quintile (Qu) ranking (right), with the patient's values superimposed on the quintile base graph. Pre Trametinib, HAIR was in the highest quintile as defined in 2013. After 10 months HAIR was in the lowest quintile as defined in 2013.

FIG. 5: Pulmonary AVMs. Thoracic CT scan images of pulmonary arteriovenous malformations (AVMs) before and after treatment. A Comparison of pulmonary AVMs across the 11 month period incorporating 10 months of Trametinib treatment. The main pulmonary AVMs present prior to Trametinib treatment were numbered #1 through #11. Individual pulmonary AVMs were compared between scans 1 month before Trametinib, and 11 months later, after 10 months of Trametinib treatment. No pulmonary AVMs improved (i.e. none became smaller), and 4 were possibly slightly larger. Error bars indicate mean and standard deviation for the scale. B Comparison of pulmonary AVMs across the 11 month period incorporating 10 months of Trametinib treatment (“post treatment”) compared to the preceding 2 years “Pre treatment”. For each pair of sequential scans, individual pulmonary AVMs were assigned to a scale of-1 (possibly smaller), 0 (no change) and +1 (possibly larger). No pulmonary AVMs improved in the 2 years pre-treatment, and again, some were marginally larger. Error bars indicate mean and standard deviation. C Representative CT scan images capturing 5 of the 11 main PAVMs pre treatment (−1 month, left panel), and after 10 months Trametinib treatment (+10 months, right panel). Of the 11 pulmonary AVMs, numbers #1-#6, #7, #8 and #11 were visible on three horizontal slices of the pre-treatment scans, and are annotated for clarity. Pulmonary AVM #7* was cited as an example of an enlarging pulmonary AVM in the blinded analyses. The +10 months slices are nearest adjacent to those in the pre-treatment series, noting these are not identically sited, and that AVM size is assessed volumetrically in three dimensions, not two. NB: The CT scans represent bone and blood vessels as white; soft tissues as grades of grey; the air-filled lungs as dark grey, and pure air (external to the body, or in the windpipe (trachea)) as black.

FIG. 6: Canonical and non-canonical BMP responses in HHT and control endothelial cells (13 cultures of BOECs: 4 controls, and 9 from the HHT genotypes ACVRL1+/− and SMAD4+/). evaluated by RNASeq. Data were RBM45-normalised which normalises for PTC-induced stress. A Canonical pathway: Normally, BMPs increase expression of ID1 by the canonical pathway. The mean alignments to ID1 (expression increased by canonical BMP signalling) were 13,958 (SD 1,986) in control BOECs and 10,907 (SD 3,037) in ACVRL1^{+/-} and ENG^{+/-} BOECs, i.e. the HHT BOEC alignments were reduced to 78% of control alignment. B Non-canonical pathway: Normally, BMPs promote post-transcriptional processing of primary miR-21 transcript (pri-miR-21) into precursor miR-21 (pre-miR-21) by non-canonical pathways. The mean unique exact alignments to mature 3′ miR-21 were 13,520 (standard deviation 2,194) in control BOECs and 8,232 (SD 1061) in ACVRL1^{+/-} and ENG^{+/-} BOECs, i.e. the HHT BOEC alignments were reduced to 60% of control alignments, with similar patterns exhibited for alignments calculated in slightly less specific ways. Bars: mean and standard deviation; circles: individual cultures.

FIG. 7: A-L RNASeq data in HHT and control endothelial cells for all 17 inhibitors of the TGF-B/BMP pathways described by Miyazawa (2017) from 16 cultures of blood outgrowth endothelial cells (BOECs: 4 controls, and 12 from the HHT genotypes ACVRL1^{+/-}, ENG^{+/-} and SMAD4^{+/-}). Data were RBM45-normalised. Bars: mean and standard deviation; circles: individual cultures. Displayed p values were calculated by Dunn's test post Kruskal Wallis (*p<0.05, **p<0.01, ns not significant). M RNASeq alignments to all 15 MAP3K genes expressed in endothelial cells in HHT BOECs compared to control BOECs. Data were DeSeq-normalised using the 8 least variable of the GC<0.15 GINI housekeeper genes in BOECs (HNRNPK, COX411, IK, UBR2, UBE2Q1, SNW1, TXNL1, and KAT5). Note MAP3K1 encoding MEKK14 is the only highly expressed MAP3K increased in HHT BOECs.

FIG. 8: MAP3K1 RNASeq data in HHT and control endothelial cells from 16 cultures of blood outgrowth endothelial cells (BOECs: 4 controls, and 12 from the HHT genotypes ACVRL1^{+/-}, ENG^{+/-} and SMAD4^{+/-}). Data were RBM45-normalised. A Untreated BOECs (N=8). B All BOECs (N=16), untreated (unRx), and treated (Rx) by BMP9 for 1 hour. Bars: mean and standard deviation; circles: individual cultures. Displayed p values were calculated by Dunn's test post Kruskal Wallis (*p<0.05).

FIG. 9: RNASeq data in HHT and control endothelial cells for MAP2K1 encoding MEK1 from 16 cultures of blood outgrowth endothelial cells (BOECs: 4 controls, and 12 from the HHT genotypes ACVRL1^{+/-}, ENG^{+/-} and SMAD4^{+/-}). Data were RBM45-normalised which normalises for PTC-induced stress. A All BOECs, B Control and ENG BOECs; C Control and ENG BOECs untreated (unRx) and treated (Rx) by BMP9 for 1 hour. Bars: mean and standard deviation; circles: individual cultures. Displayed p values were calculated by Dunn's test post Kruskal Wallis (*p<0.05).

FIG. 10: RNASeq data in HHT and control endothelial cells for MAP2K2 encoding MEK2 from 16 cultures of blood outgrowth endothelial cells (BOECs: 4 controls, and 12 from the HHT genotypes ACVRL1^{+/-}, ENG^{+/-} and SMAD4^{+/-}). Data were RBM45-normalised which normalises for PTC-induced stress. A All BOECs, B Control and ENG BOECs; C Control and ENG BOECs untreated (unRx) and treated (Rx) by BMP9 for 1 hour. Bars: mean and standard deviation; circles: individual cultures. Displayed p values were calculated by Dunn's test post Kruskal Wallis (*p<0.05).

FIG. 11: Rankings following DeSeq2-normalisation, of all ‘housekeeper’ genes which display the least variation across human datasets (as defined by a GINI-coefficient <0.15) across the 16 blood outgrowth endothelial cell (BOEC) cultures. The BOECs are ordered by % of PTC persistence (0 in control, 8-22% in HHT BOECs), C control; E ENG^{+/PTC}; A ACVRL1^{+/PTC}; S SMAD4^{+/PTC}. The y axis scale ranks from highest expression (16) to lowest expression (1), for example the highest (16) rank for NXF1 expression was in a control BOEC, whereas the lowest (1) rank for RBM45 was found in a control BOEC. Note the switch between 14 and 11% persistence for NXF1 (thick grey line, open triangles) and RBM45 (thick black line and circles).

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition.

It should be understood that this invention is not limited to the particular methodology, protocols, and reagents, etc., described herein and as such can vary. In particular, any methods and materials similar or equivalent to those described herein can be used in the practice or testing of embodiments of this disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

A “fragment” of a polypeptide typically comprises at least 50%, at least 60%, at least 70%, at least 80%, at least 90%, at least 95%, at least 97% or more of the original polypeptide.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

It must be noted that as used herein and in the appended claims, the singular forms “a”, “an”, and “the” include plural referents unless the context clearly dictates otherwise. Thus, for example, reference to “a MEK1 inhibitor” includes a plurality of such candidate agents and reference to “the MEK1 inhibitor” includes reference to one or more MEK1 inhibitors and equivalents thereof known to those skilled in the art, and so forth. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein the terms “inhibitor” and “antagonist” are used interchangeably to refer to a molecule that interferes with the expression, activity or binding of another molecule. Inhibitors may be direct/competitive (e.g. competing for the one or more binding sites of an agonist, but does not induce an active response) or indirect/non-competitive (e.g. by binding to allosteric sites).

The term “vascular anomaly” as used herein refers to a localised defect in blood vessels that can affect any part of the vasculature (capillaries, arteries, veins, lymphatics or a combination of these). Such defects are often characterized by an increased number of vessels, vessels that are both enlarged and sinuous, and vessels providing aberrant connections between different parts of the vascular network. Some vascular anomalies are congenital and therefore present at birth, others appear within weeks to years after birth and others are acquired, for example by trauma or during pregnancy. Inherited vascular anomalies have been described and are often present with a multiplicity of lesions that may increase with patients' age. Vascular anomalies can also be a part of a syndrome. Common forms of vascular anomaly include haemangioma, kaposiform haemangioendothelioma, pyogenic granuloma, capillary malformation, lymphatic malformation, venous malformation and arteriovenous malformation.

Vascular malformation is a collective term for different disorders of the vasculature (errors in vascular development). It can be a disorder of the capillaries, arteries, veins and lymphatic vessels or a disorder of a combination of these (lesions are named based on the primary vessel(s) that is/are malformed). A vascular malformation consists of a cluster of deformed vessels, due to an error in vascular development (dysmorphogenesis). Vascular malformations can be divided into slow-flow, fast-flow and complex-combined types.

The term “telangiectasia” as used herein refers to small, dilated blood vessels, typically located near the surface of the skin or mucous membranes.

The term “arteriovenous malformation” or “AVM” as used herein refers to a sizable abnormal connection between arteries and veins, bypassing the capillary system. By definition, AVMs are fast-flow malformations, as they comprise an arterial component. AVMs are distinct from telangiectasia, and a reference to telangiectasia does not encompass AVM, or vice versa.

An individual can be one who has been previously diagnosed with or identified as suffering from or having a condition in need of treatment or one or more complications related to such a condition, and optionally, have already undergone treatment for a condition as defined herein or the one or more complications related to said condition. Alternatively, an individual can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

An “individual in need” of treatment for a particular condition can be an individual having that condition, diagnosed as having that condition, or at risk of developing that condition.

The terms “subject”, “individual” and “patient” are used interchangeably herein to refer to a mammalian individual. An “individual” may be any mammal. Generally, the individual may be human; in other words, in one embodiment, the “individual” is a human. A “individual” may be an adult, juvenile or infant. An “individual” may be male or female.

The term “pharmaceutically acceptable” as used herein means approved by a regulatory agency of the Federal or a state government, or listed in the U.S. Pharmacopeia, European Pharmacopeia or other generally recognized pharmacopeia.

The publications discussed herein are provided solely for their disclosure prior to the filing date of the present application. Nothing herein is to be construed as an admission that such publications constitute prior art to the claims appended hereto. All references cited in this specification are herewith incorporated by reference with respect to their entire disclosure content and the disclosure content specifically mentioned in this specification.

### Hereditary Haemorrhagic Telangiectasia (HHT)

HHT, also known as Osler-Weber-Rendu syndrome, is one of the most common disorders (affecting approximately 1 in 5000 individuals) to be inherited as an autosomal dominant trait, and is caused by heterozygous loss-of-function mutations in one of ACVRL1, ENG, SMAD4 or GDF2. Type 1 HHT (HHT1) is caused by mutations in ENG. Type 2 HHT (HHT2) is caused by mutations in ACVRL1. HHT-juvenile polyposis syndrome (JP-HHT) is caused by mutations in SMAD4. HHT caused by GDF2 mutations (GDF2-HHT) has more recently been identified, and yet to be classified a subtype.

HHT leads to the development of vascular anomalies. In particular, HHT is characterised by severe recurrent nasal and gastrointestinal bleeding with associated anaemia, and visible dilated small blood vessels (telangiectasia) on the lips and fingertips. The majority of HHT patients are also affected by larger arteriovenous malformations (AVMs) in the pulmonary, hepatic, cerebral, pancreatic, spinal, and other circulations.

The present invention relates to the treatment of telangiectasia associated with HHT. The treatments provided by the invention may have no or little clinical effect on AVMs associated with HHT. Therefore, the present invention may be used to treat a subset of HHT patients, as described in more detail below.

The invention may relate to the treatment of HHT-associated telangiectasia, HHT-associated haemorrhage and/or HHT-associated anaemia in patients with any pathogenic variant which cases HHT (e.g. a mutation in the ACVRL1, ENG, SMAD4 or GDF2 genes). In some embodiments, the invention relates to the treatment of HHT-associated telangiectasia, HHT-associated haemorrhage and/or HHT-associated anaemia in patients with a mutation in the ACVRL1, ENG, or GDF2 genes, particularly the ENG gene. Any and all disclosure herein in relation to HHT-associated telangiectasia, HHT-associated haemorrhage and/or HHT-associated anaemia applies equally and without reservation to HHT-associated telangiectasia, HHT-associated haemorrhage and/or HHT-associated anaemia wherein the patient has a mutation in any gene associated with HHT, i.e. any of the ACVRL1, ENG, SMAD4 or GDF2 genes. By way of non-limiting example, any and all disclosure herein in relation to HHT-associated telangiectasia, HHT-associated haemorrhage and/or HHT-associated anaemia applies equally and without reservation to HHT-associated telangiectasia, HHT-associated haemorrhage and/or HHT-associated anaemia wherein the patient has a mutation in one or more of the ACVRL1, ENG, or GDF2 genes. In particular, any and all disclosure herein in relation to HHT-associated telangiectasia, HHT-associated haemorrhage and/or HHT-associated anaemia applies equally and without reservation to HHT-associated telangiectasia, HHT-associated haemorrhage and/or HHT-associated anaemia wherein the patient has a mutation in the ENG gene.

### Telangiectasia

Telangiectasia are dilated blood vessels located near the surface of the skin or mucous membranes. Telangiectasia are distinct from AVMs in a number of key mechanistic, developmental and structural characteristics. The present invention is based on the inventors' surprising identification of MEK2 inhibitors as an effective treatment for HHT telangiectasia, without a corresponding effect on HHT AVMs.

A significant difference between telangiectasia and AVMs is their size. Telangiectasia are small blood vessels, which typically do not exceed a few mm in diameter, and often measure between about 0.5 mm to about 1.0 mm in diameter. In contrast, AVMs are large vessel malformations. Even a small AVM may be ≥3 cm in diameter, and a large AVM may be ≥6 cm in diameter.

The structure of the vessels in telangiectasia and AVMs also differs. Telangiectasia typically form from the dilatation of post-capillary venules. Therefore the vessels in telangiectasia typically consist of an endothelial cell tube and a basement membrane, with occasional pericytes. In contrast, AVMs form from larger vessels, and so may comprise adventitial, smooth muscle and/or elastin tissue.

A further difference between telangiectasia and AVMs is their location within the body. Telangiectasia tend to be present in the skin and mucosal tissues (particularly the nose and gastrointestinal (GI) tract, with telangiectasia being found, from highest to lowest frequency in the nose, stomach, small intestine and colon), though can also be present in other vascular beds. In contrast, AVMs tend to be located centrally within the circulation of organs of the body, such as in the lung (pulmonary AVMs), liver (hepatic AVMs), brain (cerebral AVMs), pancreas (pancreatic AVMs) and spinal cord (spinal AVMs). The invention typically relates to the treatment of nasal telangiectasia, GI telangiectasia, nasopharyngeal telangiectasia, endobronchial telangiectasia and/or tongue telangiectasia, particularly nasal telangiectasia.

The development of telangiectasia also differs from that of AVMs. Telangiectasia tend to be sparse pre-puberty and increase in frequency with age. In contrast, AVMs are developmental, with AVM formation typically complete during childhood (e.g. cerebral AVM) or the end of puberty (e.g. pulmonary AVMs). In other words, the nasal and GI telangiectasia of HHT do not mature into AVMs.

Telangiectasia also differ mechanistically to AVMs. The initial development step of AVMs, is typically developmental (and so occurs years before a patient presents for treatment), followed in HHT by subsequent enlargement and remodelling events that result in the large vascular structures. In contrast, for telangiectasia, the triggers are typically ongoing and dynamic in nature. Thus, spontaneous regression is common for individual telangiectasia within a HHT patient, notwithstanding further development of new telangiectasia. In contrast an HHT patient's AVMs will not regress unless the blood supply to the AVM is therapeutically or pathologically disrupted.

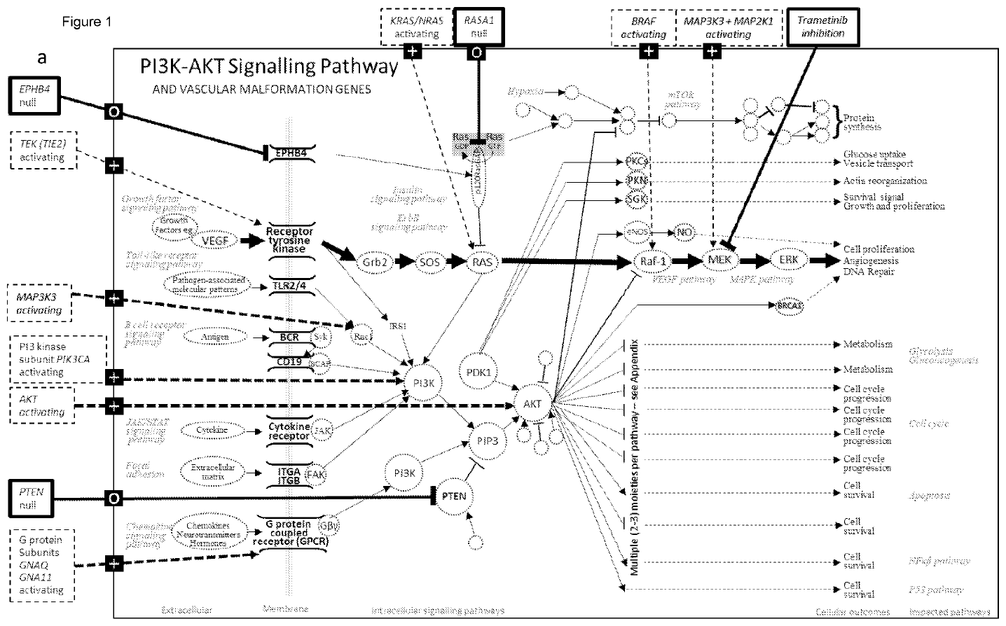

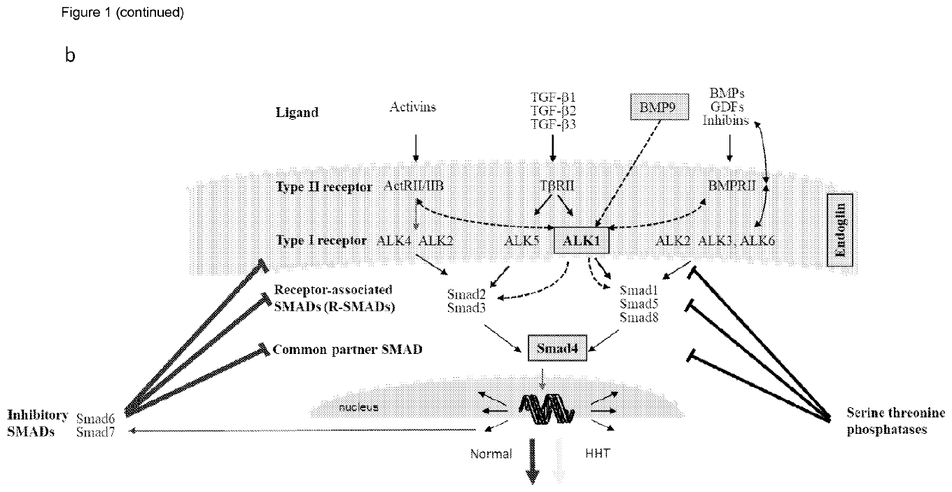

The signalling pathways involved with the formation of telangiectasia and AVMs are also different. AVMs in non-HHT settings are typically associated with dysregulation of the PI3K-AKT signalling pathway, as shown in FIG. 1A. As described and exemplified herein, the present inventor has distinguished these from the signalling pathways associated with the formation of telangiectasia in HHT, as shown in FIG. 1B. Surprisingly, given the prevailing consensus in the art, there is no overlap between these AVM-associated and telangiectasia/HHT-associated signalling pathways.

Without being bound by theory, it is believed that endothelial cell of HHT patients exhibit compensatory mechanisms such as reducing TGF-B/BMP pathway inhibitors, and increasing alternate pathways that phosphorylate the final common pathway SMAD (SMAD4). As demonstrated herein, endothelial cells of HHT patients exhibit upregulated MAP3K1 expression. It is hypothesised that this upregulated MAP3K1 expression drives SMAD4 expression to compensate for a decrease in SMAD4 signalling that would otherwise result from the aberrant TGFB/BMP9 signalling which is caused by the mutations responsible for HHT. This upregulated MAP3K1 expression also increases signalling through the MEK (particularly MEK/ERK signalling cascade, leading to endothelial cell proliferation, angiogenic sprouting and the formation of telangiectasia, and ultimately to HHT-associated haemorrhage and anaemia. Stress and/or injury can lead to dynamic increases in MAP3K1 expression, which can further increase signalling through the MEK/ERK signalling cascade, exacerbating the effects on telangiectasia, which explains the dynamic nature of the telangiectatic aspect of HHT compared with AVMs.

In sum, telangiectasia and AVMs in HHT differ structurally, developmentally, histologically and mechanistically. Therefore, these two types of vascular malformations can be considered independently, and targeted for treatment separately. The present invention provides for the treatment and/or prevention of telangiectasia associated with HHT. The methods and uses of the invention may have no impact on HHT-associated AVMs, as exemplified herein. The effect (or lack thereof) of a MEK1/2 inhibitor of the invention on AVMs may be determined and/or quantified using standard techniques such as CT scanning, MRI scanning or MRA scanning.

### Haemorrhage and Anaemia

Telangiectasia in HHT are prone to haemorrhage and bleeding, typically because of inadequate wall structures and/or high perfusion pressures. As higher concentrations of telangiectasia are found in the nasal vasculature and GI tract, haemorrhage may occur into the relatively open spaces of the nasal cavity/nostrils/atmosphere, or gastrointestinal tract. Haemorrhage of telangiectasia in the nasal vasculature typically results in nose bleeds (epistaxis). Haemorrhage of telangiectasia within the GI tract may be occult, and so manifest primarily via anaemia. It is only in very occasional patients that haemorrhage of telangiectasia within the GI tract results in clinically overt bleeding (melaena, haematemesis). More commonly, it results in black or bloody stool.

Haemorrhage has immediate overt consequences of blood loss (bleeding, light-headedness, etc.). Despite this, haemorrhage may be tolerated acutely, with compensatory mechanisms involving vasoconstriction to maintain blood pressure; restoration of circulating blood volume by salt and water retention, and replacement of the lost red blood cells containing haemoglobin via bone marrow release of reticulocytes and enhanced haemoglobin synthesis. However, chronic haemorrhage, such as occurs at telangiectasia in HHT, depletes the body's intracellular iron stores. The body is then unable to produce haemoglobin and/or erythrocytes (red blood cells, RBCs) sufficiently quickly to replace those being lost by haemorrhage. This results in the patient developing anaemia, particularly iron deficiency anaemia, which can cause microcytic anaemia (in which RBC volume is decreased) and usually accompanied by hypochromic anaemia (in which RBCs contain lower levels of haemoglobin and so are paler than normal).

In anaemia, haemoglobin levels are reduced, such that a patient's arterial oxygen content (CaO₂) cannot be maintained at normal physiological levels. When CaO₂ decreases to such a level that tissue oxygen delivery is impaired, the body responds by increasing cardiac output, by increasing heart rate and/or stroke volume, both of which require increased cardiac effort. Accordingly, symptoms of anaemia can include one or more of fatigue, weakness, shortness of breath, heart palpitations, heart failure, dizziness, impaired cognitive function; reduced immunity, impaired skeletal muscle and thyroid function, pale skin, cold hands and feet, headaches, tinnitus, hair loss, prematurity, poor maternal and perinatal outcomes in pregnancy, poor surgical outcomes including death and complications, and impaired motor and cognitive development in children Other sequalae of iron deficiency can include irreversible deficits in cognition, motor function and behaviour for neonates; more “sticky” blood via changes in platelets and coagulation factor VIII, in turn associated with ischaemic strokes and deep venous thromboses respectively; and elevated blood levels of lead (Pb²⁺) and the carcinogen cadmium (Cd²⁺) which accumulates in cells; perturbation of iron dependent enzymes and cellular pathways that are critical for DNA synthesis, for the brain in the neuronal processes of myelination, energy and neurotransmitter metabolism, and for bactericidal activity of macrophages, to name but a few.

Anaemia may be defined as a haemoglobin level below the normal for age and sex. Normal haemoglobin values in an adult are typically between about 120 to about 180 g/L (12 to 18 g/dL), but are influenced by the age, sex, ethnic origin and activity of the person. NICE defines anaemia as a haemoglobin (Hb) level two standard deviations below the normal for age and sex:

In men aged over 15 years—Hb below 130 g/L.

In non-pregnant women aged over 15 years—Hb below 120 g/L.

In children aged 12-14 years—Hb below 120 g/L.

In pregnant women—Hb below 110 g/L throughout pregnancy.

Postpartum—below 100 g/L.

Thus, the invention may relate to the treatment of patients who may be classified as anaemic according to such standard clinical definitions based on haemoglobin levels. Alternatively or in addition, since iron deficiency itself results in symptoms, some HHT patients with severe bleeding may be symptomatic even at relatively “normal” haemoglobin levels. As such, the invention may relate to the treatment of HHT patients identified as anaemic based on criteria other than haemoglobin levels, such as the severity of their anaemia or anaemia-related symptoms. Thus, the invention may relate to the treatment of HHT patients presenting with anaemia, or HHT patients for whom anaemia is avoided only through invasive treatments such as blood transfusions, intravenous iron or others, as described herein.

The present inventors have previously demonstrated that some HHT patients with severe and/or recurrent haemorrhage anaemia is exacerbated by reduced intravascular erythrocyte survival (haemolysis). When present, haemolysis is typically observed late in HHT disease progression, and in patients receiving regular i.v. iron replacement and/or blood transfusions. Typically in this setting, there are more macrocytic red cell indices, in which RBC volume is increased, reflecting release of larger, less mature RBC precursors from the bone marrow.

The present invention provides for the treatment and/or prevention of HHT-induced anaemia and/or telangiectasia. This encompasses the treatment and/or prevention of one or more symptom of HHT-induced anaemia and/or telangiectasia. This also encompasses the treatment of anaemia resulting from haemolysis. In some embodiments, the invention relates to the treatment of patients who suffer from chronic or recurrent haemorrhage, such as nose bleeds and/or GI haemorrhage. As the frequency, duration and/or severity of haemorrhage can vary significantly, what constitutes chronic or recurrent haemorrhage may vary between patients, but can in all cases be readily determined by a medical practitioner, who will therefore be able to identify patients suitable for treatment. By way of non-limiting example, recurrent haemorrhage (e.g. nose bleeds) may be defined as haemorrhage (e.g. nose bleeds) occurring infrequently (less than once a week), at least once a week, at least twice a week, at least three times a week, every other day, or at least once a day. Alternatively or additionally, recurrent haemorrhage (e.g. nose bleeds), when they occur, may be very short (last less than 5 minutes) or last for at least 5 minutes, at least 10 minutes, at least 15 minutes, at least 30 minutes, at least 1 hour or more. For example, recurrent haemorrhage (e.g. nose bleeds), when they occur, may last for between about 5 minutes to about 1 hour or more; between about 5 minutes to about 30 minutes, etc.

As described herein, a standard first-line treatment for anaemia, including HHT-associated anaemia is dietary iron and/or oral iron supplements. Recommended daily iron intake is typically in the range of from about 7 mg to about 15 mg, encompassing the recommended daily intake of iron of about 7 mg to about 10 mg (e.g. about 8.7 mg) for an adult man, or from about 10 mg to about 15 mg (e.g. about 14.8 mg) for an adult woman. Oral iron tablets used in the treatment of anaemia usually contain at least 35-65 mg of elemental iron and are commonly prescribed up to three times per day, when gastrointestinal side effects are commonly dose-limiting, and iron-induced haemorrhage counterproductive in approximately 1 in 20 people with HHT. Conventionally, HHT patients who are non-responsive to such treatments, typically require more invasive treatments, such as treatments comprising intravenous iron infusions (when iron-induced haemorrhage is counterproductive in approximately 1 in 10 people with HHT), surgery, anti-angiogenic agents and/or blood transfusion. Non-limiting examples of anti-angiogenic agents used for to treat HHT patients who are non-responsive to dietary iron and/or oral iron supplements include bevacizumab and thalidomide, with tyrosine kinase inhibitors (e.g. sorafenib, pazopanib (and analogue GW771806)), nintedanib, anti-AngPT2 antibodies (e.g. sunitinib), buparlisib (anti PI3K) and immunosuppressive agents (e.g. tacrolimus, and sirolimus)) under research evaluations.

Accordingly, in some preferred embodiments, the invention relates to the treatment of telangiectasia, such as the treatment or prevention of HHT-associated anaemia and/or haemorrhage, where symptoms and evidence of iron deficiency and/or anaemia persist despite dietary iron and/or oral iron supplements, as described herein. Thus, the present invention provides an alternative to conventional, invasive HHT treatments, such as treatments comprising surgery, anti-angiogenic agents and/or blood transfusion.

### MAP2K1/2 (MEK1/2)

Mitogen-activated protein kinases (MAPKs) are a superfamily of protein kinases that are activated by diverse stimuli via protein kinase cascades. They are the final components of the cascades, activated by phosphorylation by mitogen-activated protein kinase kinases (MAP kinase kinases, MAPKKs), which in turn are activated by mitogen-activated protein kinase kinase kinases (MAP kinase kinase kinases, MAPKKKs).

Dual specificity mitogen-activated protein kinase kinases 1 and 2, (MAP2K1/2, also referred to interchangeably as MEK1/2) are an essential component of the MAP kinase signal transduction pathway. MEK1/2 lie upstream of extracellular signal-regulated kinases (ERKs) which are the classical MAP kinase. MEK1/2 act as gatekeepers since uniquely they are threonine tyrosine kinases and are the only kinases able to phosphorylate ERK1 and ERK2, on both Thr 202 and Tyr 204, with both phosphorylations required to activate ERKs. In turn, ERK 1 and ERK2 are the only known substrates of MEK1/2. MEK1 and MEK2 stimulate ERK1/2 MAP kinase activity upon activation by a wide variety of extra- and intracellular signals. For example, MEK1 and MEK2 may be activated by an upstream RAS/RAF cascade, DNA damage, or oxidative stress. Activation of MEK1 typically occurs through phosphorylation of Ser-218 and Ser-222. MEK1 is also the target of negative feed-back regulation by its substrate kinases, such as MAPK1/ERK2. These phosphorylate MAP2KI/MEK1 on Thr-292, thereby facilitating dephosphorylation of the activating residues Ser-218 and Ser-222.

An exemplary human MEK1 nucleic acid sequence is NCBI Accession No. NM_002755 (version 4, i.e. NM_002755.4, accessed 22 Jan. 2022), comprised herein as SEQ ID NO: 1. An exemplary human MEK1 protein sequence is UniProt Accession No. Q02750 (sequence version 2, accessed 22 Jan. 2022), comprised herein as SEQ ID NO: 2. An exemplary human MEK2 nucleic acid sequence is NCBI Accession No. NM_030662 (version 4, i.e. NM_030662.4, accessed 22 Jan. 2022), comprised herein as SEQ ID NO: 3. An exemplary human MEK1 protein sequence is UniProt Accession No. P36507 (accessed 22 Jan. 2022), comprised herein as SEQ ID NO: 4.

Inhibition of MEK1 and/or MEK2

In some embodiments, the present invention relates to the use of compounds to inhibit the action of MEK1 and/or MEK2 (abbreviated herein as MEK1/2), i.e. compounds which inhibit MEK1/2 activity. MEK1/2 activity may be inhibited by any appropriate means. Suitable standard techniques are known in the art. Inhibition may take place via any suitable mechanism, depending for example on the nature (see below) of the compound used, e.g. steric interference in any direct or indirect interaction or inhibition of MEK1/2. In the context of the present invention a MEK1/2 inhibitor (interchangeably referred to herein as a MEK1/2 antagonist) is any compound which inhibits, decreases, suppresses or ablates the action of MEK1/2, whether in part or completely. The invention may relate to the inhibition of MEK1 (i.e. treatment with MEK1 inhibitors). The invention may relate to the inhibition of MEK2 (i.e. treatment with MEK2 inhibitors). The invention may relate to the inhibition of MEK1 and MEK2 (i.e. treatment with inhibitors of both MEK1 and MEK2).

A decrease in MEK1/2 activity may be measured relative to a control. Thus, the activity of MEK1/2 in a sample of endothelial cells, or endothelial precursor or progenitor cells, or in a sample obtained from an individual/patient to be treated according to the invention may be compared with the activity of MEK1/2 in a control. Activity may be quantified in any appropriate terms, for example phosphorylation of Ser-218 and/or Ser-222 of MEK1/2, binding of MEK1/2 to a downstream MEK1/2 target, phosphorylation of a downstream MEK1/2 target, or in terms of MEK1/2 expression as defined herein. Any appropriate technique or method may be used for quantifying MEK1/2 activity. Suitable techniques are known in the art.

Typically the control is an equivalent population or sample in which no MEK1/2 inhibitor has been added, for example a sample obtained from a different individual to which the compound has not been administered, or the same individual the prior to administration of the compound. Conventional methods for the treatment of HHT or HHT-associated telangiectasia, including known methods may be considered control methods according to the present invention. Thus, a control may be a patient with HHT (the same patient or another patient). Alternatively, a control may be an individual without HHT, such as a healthy individual.

In the context of the present invention, a reference to inhibiting MEK1/2 activity may be understood to mean that, the activity of MEK1/2 is decreased by at least 10%, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, up to total (100%) inhibition of MEK1/2 activity, as compared with the control. Typically MEK1/2 activity is decreased by at least 50%, preferably at least 70%, more preferably at least 80%, more preferably at least 90%, even more preferably at least 95% or more compared with the control.

The activity of MEK1/2 may be determined by quantitative and/or qualitative analysis, and may be measured directly or indirectly.

The activity of MEK1/2 relative to a control may be determined using any appropriate technique. Suitable standard techniques are known in the art.

The activity of MEK1/2 may be inhibited compared with a control for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week. Preferably, the activity of MEK1/2 is decreased for at least 12 to 72 hours. MEK1/2 may be inhibited indefinitely. Typically this is assessed relative to the last administration of the MEK1/2 inhibitor.

In some embodiments, MEK2 activity may be inhibited according to the invention. Thus, a MEK1 inhibitor as described herein may inhibit MEK2 in addition or alternatively to MEK1. Any and all disclosure herein in relation to inhibition of MEK1 applies equally and without reservation to inhibition of MEK2. Reference herein to inhibition of MEK1/2 refers to inhibition of MEK1 and/or inhibition of MEK2.

### MEK1/2 Inhibitors

Prior to the present application, the consensus in the art teaches that HHT endothelial cells behave normally in most settings. Importantly, however, the present inventors are the first to appreciate that, whilst HHT endothelial cells may be able to compensate for steady state conditions, they are not able to compensate for dynamic signals. Without being bound by theory, the inventors are the first to appreciate that HHT-associated telangiectasis develop as a result of short-term or dynamic upregulation of ERK1/2 signalling in response to stress upregulation of MAP3K1 (MEKK1). MAP3K1 can be induced by reactive oxygen species (ROS) associated with stress, resulting in increased MEK1/MEK2 signalling and hence increased ERK1/ERK2 signalling. A typically recognised consequence of MEK1/2 activation is cellular senescence (both through DNA-damage linked and DNA damage-independent induction of p21), while persistent activation results in oxidative stress that creates a positive feedback loop. The role of ROS in inducing MAP3K1 signalling explains clinical observations of injury, inflammation and iron-induced exacerbations (even at low doses of iron supplements) of HHT haemorrhage and/or anaemia in HHT patients.

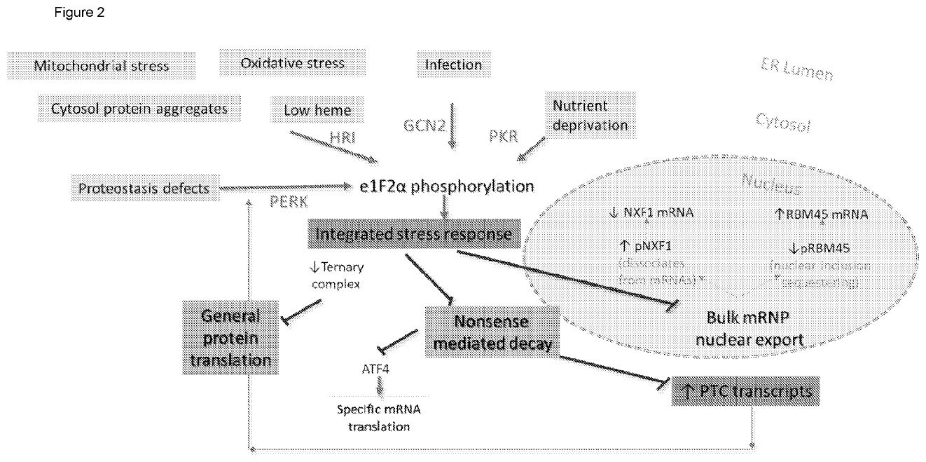

All cells respond to stress via an integrated stress response (ISR). As shown in FIG. 2, different types of stress such, as mitochondrial stress, oxidative stress, infection, nutrient deprivation, low heme, etc., result in translation initiation factor 2 alpha (elF2a) phosphorylation. Phosphorylation of elF2a results in the inhibition of translation-dependent nonsense mediated decay (NMD), which in turn allows transcripts harbouring premature termination codons (PTCs) to persist, resulting in stressed cells having a protein burden, which further stresses the cell and creates a positive feedback loop. This is consistent with the fact that, as exemplified herein, endothelial cells from HHT patients exhibit higher levels of PTC-harbouring transcripts compared with normal endothelial cells. Furthermore, the ISR can also result in phosphorylation and inactivation of SMAD4, again creating a positive feedback loop, as discussed below (compensating for reduced SMAD4 signalling by increasing MAP3K1 signalling also drives an increase in MEK/ERK signalling). Whilst short-term stressors, or stressors which can be readily identified, can be addressed clinically by removal of the stressor, for other long-term stressors, or other stressors which cannot be readily identified or removed, it would be clinically advantageous to have an alternative approach to mitigate the effect of the stressors, and hence treat or prevent telangiectasia associated with HHT, and particularly HHT induced anaemia and/or haemorrhage. The present invention has potential clinical utility in treating or preventing HHT, particularly HHT induced anaemia and/or haemorrhage, associated with any stress, such as the stressors identified herein, and particularly those illustrated in FIG. 2. Treatment or prevention according to the present invention may reduce PTC persistence compared with a control (e.g. PTC persistence in the same patient prior to treatment according to the invention, or an untreated HHT patient). By way of non-limiting example, treatment or prevention according to the invention may reduce endothelial HHT gene ACVRL1, ENG and SMAD4, and wider PTC persistence by at least 5%, at least 10%, at least 15%, at least 20%, at least 25% or more compared with a control. Alternatively or in addition, treatment or prevention according to the present invention may reduce endothelial HHT gene ACVRL1, ENG and SMAD4 and wider PTC persistence, such that the level of PTC persistance is reduced towards a baseline level (e.g. PTC persistence in a healthy individual).

As illustrated herein, Nuclear RNA Export Factor 1 (NXF1) RNA levels are reduced in stress conditions when unbound protein levels rise following dissociation from mRNAs, and RNA Binding Motif Protein 45 (RBM45) RNA levels are increased in stress conditions to restore levels of protein when bound to m6A-modified mRNAs in reversible nuclear inclusions. Therefore, treatment or prevention according to the present invention may increase RNA levels of NXF1 and/or decrease RNA levels of RBM45. The RNA level of NXF1 may be increased by at least 5%, at least 10%, at least 15%, at least 20%, at least 25% or more compared with a control. Alternatively or in addition, the RNA level of RBM45 may be decreased by at least 5%, at least 10%, at least 15%, at least 20%, at least 25% or more compared with a control.

As discussed above, BMP9 is a causal ligand for HHT, and particularly HHT telangiectasia, with a loss of or reduction in BMP9 signalling resulting in a decrease in SMAD4 signalling. Cells in HHT patients compensate for this reduced SMAD4 signalling by increasing MAP3K1 signalling, which can increase SMAD4 signalling independently of BMP9. However, increased MAP3K1 signalling also drives an increase in MEK/ERK signalling, resulting in telangiectasia and HHT-associated haemorrhage and anaemia. Previous work by third parties have suggested treatment using BMP9. However, as (i) the BMP9/TGFB signalling pathway is dysfunctional in HHT patients and (ii) BMP9 typically circulates in excess in a physiological setting, administration of exogenous BMP9 may not be beneficial in the treatment of HHT-associated telangiectasia, haemorrhage and anaemia. As exemplified herein, the present inventors are the first to demonstrate that a MEK1/2 inhibitor decreases HHT telangiectasia, and the associated haemorrhage and anaemia. The present inventors are the first to appreciate that, out of the hundreds of cellular responses to changes in BMP9 signalling, the resulting changes to MEK1 and/or MEK2 signalling are contributory or causal to HHT telangiectasia. As such, the present inventors are the first to appreciate that HHT-associated telangiectasis can be treated by MEK1/2 inhibitors, and to support this with clinical evidence.

MEK1/2 inhibitors of the invention may be specific for MEK1 and/or MEK2. By specific, it will be understood that the compound binds to MEK1/2, with no significant cross-reactivity to any other molecule, particularly any other protein, other than MEK1/2. For example, a modulator that is specific for MEK1/2 will show no significant cross-reactivity with human neutrophil elastase. Cross-reactivity may be assessed by any suitable method. Cross-reactivity of MEK1/2 inhibitor with a molecule other than MEK1/2 may be considered significant if the inhibitor binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to MEK1/2. An inhibitor that is specific for MEK1/2 may bind to another molecule such as human neutrophil elastase at less than 90%, 85%, 80%, 75%, 70%, 65%, 60%, 55%, 50%, 45%, 40%, 35%, 30%, 25% or 20% the strength that it binds to MEK1/2. Preferably, the inhibitor binds to the other molecule at less than 20%, less than 15%, less than 10% or less than 5%, less than 2% or less than 1% the strength that it binds to MEK1/2.

MEK1/2 inhibitors of the invention may have off-target effects. An off-target effect is activity against a target other than MEK1/2. Typically compounds with off-target effects are encompassed by the present invention if the activity against the non-MEK1/2 target is not significant compared with the activity against MEK1/2. Whether an off-target effect is significant may depend on the intended use of the inhibitor. The presence and magnitude of any potential off target effects can be readily assessed using standard methods known in the art.

Any suitable MEK1/2 inhibitor may be used according to the invention, for example small molecules, PROTAC reagents, double stranded RNA (dsRNA), small interfering RNA (siRNA), small hairpin RNA (shRNA), microRNA, antisense (single stranded) RNA, peptides and peptidomimetics, antibodies, aptamers and ribozymes. Preferred inhibitors include small molecules.

### Small Molecules

Preferably small molecules may be used to inhibit MEK1/2 as described herein. As defined herein, small molecules are low molecular weight compounds, typically organic compounds. Typically, a small molecule has a maximum molecule weight of 900 Da, allowing for rapid diffusion across cell membranes. In some embodiments, the maximum molecular weight of a small molecule is 500 Da. Typically a small molecule has a size in the order of 1 nm.

Many small molecule inhibitors of MEK1/2 are known in the art, any of which may be used according to the present invention. Non-limiting examples of MEK1/2 inhibitors include trametinib (N-(3-{3-Cyclopropyl-5-[(2-fluoro-4-iodophenyl)amino]-6,8-dimethyl-2,4, 7-trioxo-3,4,6, 7-tetrahydropyrido[4,3-d]pyrimidin-1(2H)-yl}phenyl)acetamide), cobimetinib ((S)-[3,4-Difluoro-2-(2-fluoro-4-iodophenylamino)phenyl][3-hydroxy-3-(piperidin-2-yl) azetidin-1-yl] methanone), binimetinib (5-((4-bromo-2-fluorophenyl)amino)-4-fluoro-N-(2-hydroxyethoxy)-1-methyl-1H-benzo[d]imidazole-6-carboxamide), selumetinib (6-(4-bromo-2-chloroanilino)-7-fluoro-N-(2-hydroxyethoxy)-3-methylbenzimidazole-5-carboxamide), TAK-733 (one of a series of 8-methylpyrido[2,3-d]pyrimidine-4,7 (3H,8H)-dione derivatives), CH4987655, RDEA119/BAY 869766, PD-325901, CI-1040 and PD-035901.

In some preferred embodiments, a small molecule MEK1/2 inhibitor is one that can be administered by non-invasive means, particularly by oral administration. This is because it is an object of the present invention to provide effect treatments for telangiectasia associated with HHT that are less invasive than some of the current treatment options (e.g. blood transfusion and/or i.v. administration of anti-angiogenic agents). As described herein, there are numerous advantages associated with such non-invasive treatments, making this a preferred treatment option. One example of a MEK1/2 inhibitor which may be administered orally is trametinib. Therefore, in some particularly preferred embodiments, the invention relates to the treatment of HHT-associated telangiectasia, and/or haemorrhage and/or anaemia associated with HHT and HHT-associated telangiectasia using trametinib. The chemical structure of trametinib is set out below:

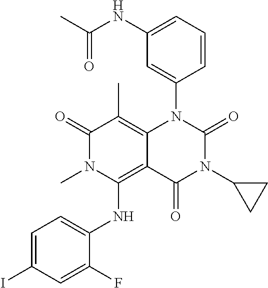 [SMILES: CC(=O)Nc1cccc(-n2c(=O)n(C3CC3)c(=O)c3c(Nc4ccc(I)cc4F)n(C)c(=O)c(C)c32)c1]

### PROTAC Reagents

Proteolysis targeting chimeras (also referred to as PROTACs or PROTAC reagents) may be used to inhibit MEK1/2 activity as described herein. PROTACs are heterobifunctional small molecules that simultaneously bind a target protein and ubiquitin ligase, enabling ubiquitination and degradation of the target. In more detail, a PROTAC reagent typically comprises a ligand for the target protein (in the case of the present invention, MEK1/2) and a ligand for an E3 ligase recognition domain. Through the use of such a PROTAC, an E3 ligase is recruited to the PROTAC-bound MEK1/2, inducing ubiquitin transfer from the E3 ligase complex to the target protein (in the case of the present invention, MEK1/2). Once the PROTAC has induced a sufficient degree of ubiquitination of the target, it is then recognised and degraded by the proteasome.

As a non-limiting example, a PROTAC reagent may be produced by conjugating a ligand for an E3-ligase to a small molecule inhibitor as described herein (preferably trametinib) via a linker. In a preferred embodiment, a PROTAC reagent comprises a ligand for the E3 RING Cullin ligase von-Hippel Lindau protein (VHL) or cereblon-a part of a CRL4 E3 RING Cullin ligase complex, connected to a small molecule inhibitor of the invention via a linker. In some particularly preferred embodiments, the PROTAC reagent comprises a ligand for the E3 RING Cullin ligase von-Hippel Lindau protein (VHL) connected to trametinib, connected via a linker. In other particularly preferred embodiments, the PROTAC reagent comprises cereblon (a part of a CRL4 E3 RING Cullin ligase complex) and trametinib, connected via a linker.

Because of their mechanism of action, PROTAC reagents simply need any ligand for the target protein. The functional pharmacology of the ligand, in the absence of the linker and E3 ligase ligand, is unimportant. Therefore in some embodiments a MEK1/2 inhibitory PROTAC reagent of the present invention may comprises a small molecule MEK1/2 agonist as the ligand.

### Double-Stranded RNA

Double-stranded RNA (dsRNA) molecules may be used to inhibit MEK1/2 activity as described herein.

Using known techniques and based on a knowledge of the sequence of MEK1/2, dsRNA molecules can be designed to antagonise MEK1/2 by sequence homology-based targeting of the corresponding RNA sequence. Such dsRNAs will typically be small interfering RNAs (siRNAs), small hairpin RNAs (shRNAs), or micro-RNAs (miRNAs). The sequence of such dsRNAs will comprise a portion that corresponds with that of a portion of the mRNA encoding MEK1/2. This portion will usually be 100% complementary to the target portion within the mRNA transcribed from the MEK1/2 gene, but lower levels of complementarity (e.g. 90% or more or 95% or more) may also be used. Typically the % complementarity is determined over a length of contiguous nucleic acid residues. A dsRNA molecule of the invention may, for example, have at least 80% complementarity to the target portion within the mRNA transcribed from the MEK1/2 gene measured over at least 10, at least 20, at least 30, at least 40, at least 50, at least 60, at least 70, at least 80, at least 90, or more nucleic acid residues, up to the dsRNA molecule having at least 80% complementarity the mRNA transcribed from the MEK1/2 gene of the invention over the entire length of the dsRNA molecule.

In a preferred embodiment, the dsRNA is a shRNA. ShRNA can be delivered to a patient or a patients cells (such as endothelial cells or endothelial cell precursors) by any appropriate means. Suitable techniques are known in the art and include the use of plasmid, viral and bacterial vectors to deliver the shRNA. Typically, the shRNA is delivered using a viral vector delivery system. In a preferred embodiment, the viral vector is a lentiviral vector.

Generally, once the shRNA has been delivered to an endothelial cell or endothelial precursor cell, it is then transcribed in the nucleus and processed. The resulting pre-shRNA is exported from the nucleus and then processed by dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both of these cases, the shRNA leads to target gene silencing.

A variant sequence may have at least 80% sequence identity to an shRNA sequence of the invention, measured over any appropriate length of sequence. Typically the % sequence identity is determined over a length of contiguous nucleic acid or amino acid residues. A variant sequence of the invention may, for example, have at least 80% sequence identity to a sequence of the invention measured over at least 10, at least 20, at least 30, at least 40, at least 50, at least 60, at least 70, at least 80, at least 90, or more nucleic acid or amino acid residues.

For example, a variant shRNA molecule of the invention may have at least 80% sequence identity with an shRNA molecule of the invention measured over at least 10, at least 20, at least 30, at least 40, at least 50, at least 60 or more nucleic acid residues, up to the variant shRNA molecule having at least 80% sequence identity with the shRNA molecule of the invention over the entire length of the variant shRNA molecule.

### Antisense RNA

Single-stranded DNA (ssDNA) molecules, also known as antisense RNA, may be used to inhibit MEK1/2 activity as described herein.

Using known techniques and based on a knowledge of the sequence of the MEK1/2 gene, antisense RNA molecules can be designed to antagonise the MEK1/2 gene by sequence homology-based targeting of the corresponding RNA. The sequence of such antisense will comprise a portion that corresponds with that of a portion of the mRNA transcribed from the MEK1/2 gene. This portion will usually be 100% complementary to the target portion within the transcribed mRNA but lower levels of complementarity (e.g. 90% or more or 95% or more) may also be used.

### Aptamers

Aptamers may be used to inhibit MEK1/2 activity as described herein.

Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

Aptamers may be generated using routine methods such as the Systematic Evolution of Ligands by Exponential enrichment (SELEX) procedure. SELEX is a method for the in vitro evolution of nucleic acid molecules with highly specific binding to target molecules. It is described in, for example, U.S. Pat. Nos. 5,654,151, 5,503,978, 5,567,588 and WO 96/38579.

The SELEX method involves the selection of nucleic acid aptamers and in particular single stranded nucleic acids capable of binding to a desired target, from a collection of oligonucleotides. A collection of single-stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

### Peptidomimetics

Peptidomimetics may be used to inhibit MEK1/2 activity as described herein.

Peptidomimetics are compounds which mimic a natural peptide or protein with the ability to interact with the biological target and produce the same biological effect. Peptidomimetics may have advantages over peptides in terms of stability and bioavailability associated with a natural peptide. Peptidomimetics can have main- or side-chain modifications of the parent peptide designed for biological function. Examples of classes of peptidomimetics include, but are not limited to, peptoids and β-peptides, as well as peptides incorporating D-amino acids.

### Antibodies

Antibodies may be used to inhibit MEK1/2 activity as described herein.

As used herein, the term antibody encompasses the use of a monoclonal antibody or polyclonal antibody, as well as the antigen-binding fragments of a monoclonal or polyclonal antibody, or a peptide which binds to MEK1/2 with specificity. The antibody may be a Fab, F(ab′)2, Fv, scFv, Fd or dAb.

### Therapeutic Indications

The invention provides a MEK1/2 inhibitor for use in a method of treating and/or preventing telangiectasia associated with HHT. The MEK1/2 inhibitor for use in said method of therapy may be any MEK1/2 inhibitor as described herein. Preferably the MEK1/2 inhibitor is one which is suitable for oral administration, particularly trametinib.

Typically the method of therapy comprises administering a MEK1/2 inhibitor (as described herein) to a patient or subject. The MEK1/2 inhibitor typically results in a reduction in MEK1/2 activity, as described herein. Said reduction in MEK1/2 activity may be quantified relative to a control, as described herein.

The activity of a MEK1/2 inhibitor may be determined by quantitative and/or qualitative analysis, and may be measured directly or indirectly.

A MEK1/2 inhibitor of the invention may elicit a decrease in the number and/or size (e.g. diameter and/or volume) of telangiectasia in a HTT patient. By way of non-limiting example, a reference to decreasing the number and/or size (e.g. diameter and/or volume) of telangiectasia may be understood to mean that, the number and/or size (e.g. diameter and/or volume) of telangiectasia is decreased by at least 10%, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, up to total (100%), as compared with the control. Typically the number and/or size (e.g. diameter and/or volume) of telangiectasia is decreased by at least 50%, preferably at least 70% inhibition or more compared with the control.

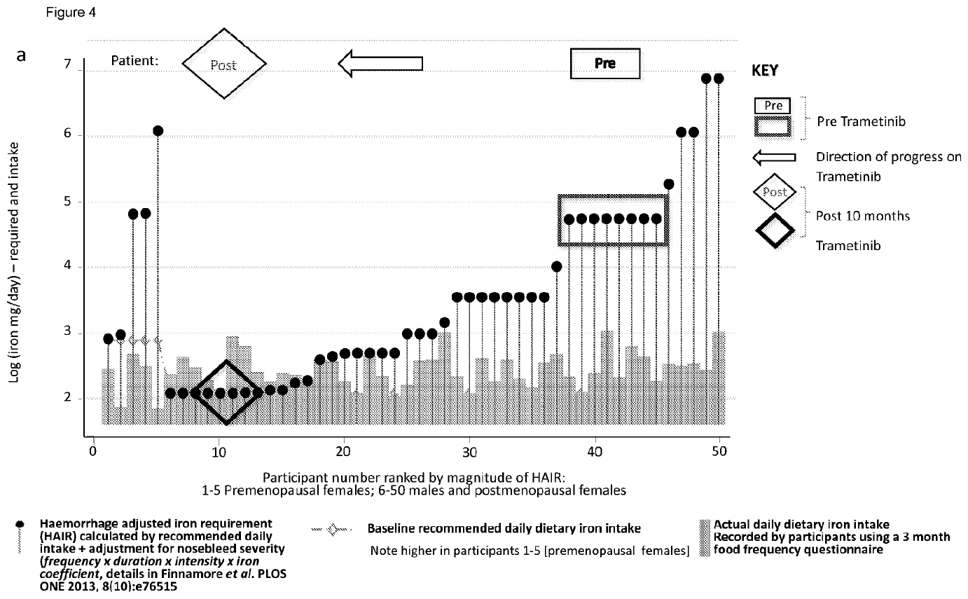

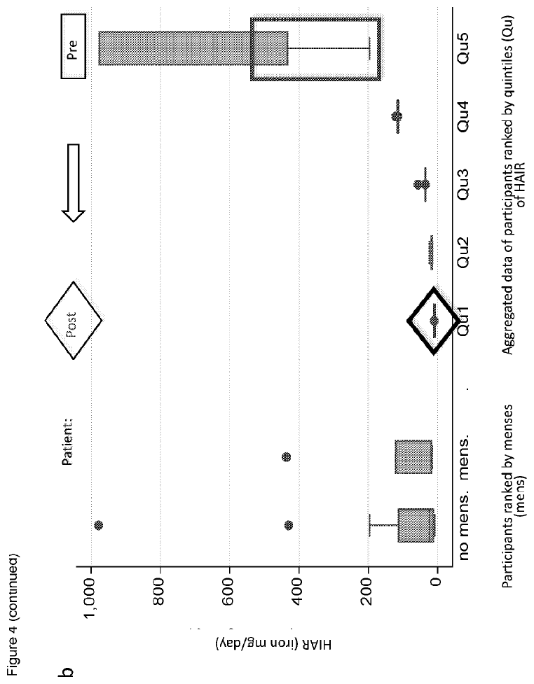

A MEK1/2 inhibitor of the invention may decrease the frequency, severity and/or duration of haemorrhages associated with telangiectasia (i.e. HHT-associated haemorrhages) in a patient, such as the average (mean) frequency (how often haemorrhage occurs), intensity (amount of blood lost), duration (how long an individual haemorrhage lasts) and/or severity of said haemorrhages. Severity of haemorrhage may be quantified as a product of the intensity, frequency and duration of haemorrhage, referred to as haemorrhage adjusted iron requirements (HAIR), as per the calculations described in Finnamore et al. (PLOS One 2013; 8 (10): e76516), which is herein incorporated by reference in its entirety) and as illustrated in FIG. 4. Typically, mild haemorrhage results in a haemorrhage adjusted iron requirement that can still be met by usual dietary daily intakes of iron, and could correspond to a short (2-5 minute), low intensity, “dripping” nosebleed once or twice a week. Typically, moderate haemorrhage results in a haemorrhage adjusted iron requirement that can be met by low dose supplements of iron (35 mg/day), and could correspond to longer (10-20 minute) and/or more frequent (several times a week) nosebleeds, still usually of low intensity (“dripping”). Typically, severe haemorrhage results in a haemorrhage adjusted iron requirement that cannot be met by tolerated doses of oral iron supplements, and could correspond to longer (>20 minute), more frequent (daily or several times a day) nosebleeds, that are usually of high intensity (“pourers” or “gushers”). By way of non-limiting example, treatment with a MEK1/2 inhibitor according to the invention may reduce haemorrhage from severe to moderate or mild, or from moderate to mild. In numerical terms, mild HAIR may be no greater than the reference nutrient intake (RNI) for iron which is 8.7 mg/day for men and postmenopausal women, and 14.8 mg/day for premenopausal women (Department of Health. Dietary reference values for food energy and nutrients for the United Kingdom. London: HMSO; 1991), and severe HAIR may not be expected to be met by oral iron of approximately 65 mg/day, with moderate HAIR falling in between (i.e. between 8.7 mg/day to 65 mg/day mg/day for men and postmenopausal women and between 14.8 mg/day to 65 mg/day mg/day for premenopausal women). Exemplary numerical HAIR values for mild/moderate/severe haemorrhage are illustrated in FIG. 4.

By way of a further non-limiting example, treatment with a MEK1/2 inhibitor according to the invention may result in a decrease in the frequency of haemorrhages (e.g. nosebleeds) to 1 a week or less, 1 every two weeks or less, 2 or 3 a month or less, 1 a month or less, or less frequently. By way of a further non-limiting example, treatment with a MEK1/2 inhibitor according to the invention may result in a decrease in the intensity of haemorrhages (e.g. nosebleeds) by at least 10%, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, up to total (100%) inhibition, as compared with the control. Typically the intensity of haemorrhages (e.g. nosebleeds) is decreased by at least 50%, preferably at least 70%, more preferably at least 80%, compared with the control. By way of a further non-limiting example, treatment with a MEK1 inhibitor according to the invention may result in a decrease in the duration of haemorrhages (e.g. nosebleeds) by at least 5 minutes, by at least 10 minutes, by at least 15 minutes, by at least 30 minutes, by at least 1 hour, or more. By way of a further non-limiting example, treatment with a MEK1 inhibitor according to the invention may result in a decrease in the severity of haemorrhages (e.g. nosebleeds) by at least 10%, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, up to total (100%), as compared with the control. Typically the severity of haemorrhages (e.g. nosebleeds) is decreased by at least 50%, preferably at least 70%, more preferably at least 80%.

A MEK1/2 inhibitor of the invention may decrease anaemia associated with telangiectasia (i.e. HHT-associated anaemia) in a patient. A decrease in anaemia associated with telangiectasia may be quantified as a decrease in one or more symptoms of anaemia, as described herein, or by an increase in the patient's haemoglobin, red cell indices, or by the improvement in iron indices e.g. blood iron, transferrin saturation index and/or ferritin levels. By way of non-limiting example, a MEK1/2 inhibitor of the invention may increase a patient's haemoglobin levels by at least 10%, at least 20%, at least 30%, at least 40%, at least 50%, at least 60, at least 70%, at least 80%, at least 85%, at least 90%, at least 95%, at least 100%, compared with a control. Typically a MEK1/2 inhibitor of the invention may increase a patient's haemoglobin levels by at least 20%, at least 50%, preferably at least 70%, more preferably at least 80% of maximum possible to achieve a normal haemoglobin. Alternatively, a MEK1/2 inhibitor of the invention may allow a patient's haemoglobin levels to be kept essentially constant (rather than being increased), but without the need for invasive treatments such as iron or blood transfusions. In some preferred embodiments, a MEK1/2 inhibitor of the invention may increase a patient's haemoglobin levels to within the normal range of haemoglobin levels (e.g. matched for age and sex). A MEK1/2 inhibitor of the invention may maintain a patient's haemoglobin at a clinically acceptable level without the need for invasive treatments such as iron or blood transfusions.

Treatment with a MEK1/2 inhibitor according to the invention may reduce the frequency with which a patient requires more invasive treatments, such as blood transfusions, iron transfusions or i.v. administration of anti-angiogenic agents. By way of a non-limiting example, treatment with a MEK1/2 inhibitor according to the invention may result in a decrease in the frequency of invasive treatment(s) (e.g. blood transfusion) required by said patient to less than 1 every 4 weeks, less than 1 every 6 weeks, less than 1 every 8 weeks, less than 1 every 12 weeks, or more. In some embodiments, treatment with a MEK1/2 inhibitor according to the invention abrogates the need for a patient requires more invasive treatments, such as blood transfusions, iron transfusions or i.v. administration of anti-angiogenic agents (such as those described herein, including bevacizumab, thalidomide).

The effects of treatment with a MEK1/2 inhibitor may be determined by quantitative and/or qualitative analysis, suitable methods and techniques are known in the art. By way of non-limiting example, size and/or number of telangiectasia may be determined by visual and endoscopic inspection; highly sensitive imaging (such as scanning by computerised tomography (CT), magnetic resonance imaging (MRI) or magnetic resonance angiography (MRA) or positron emission tomography (PET)), a patient's haemoglobin level may be quantified by any appropriate test, such as a standard clinical full blood count. By way of a further non-limiting example, data regarding the frequency, severity and/or duration of haemorrhages (e.g. nosebleeds) may be captured using tools such as the Epistaxis Severity Score; Haemorrhage Adjusted Iron Requirement (HAIR); and standard quality of life (QoL) questionnaires, such as the International Quality of Life Assessment questionnaire, version 1.1 (IQOLA 1.1).

The duration of action of a MEK1/2 inhibitor according to the invention may be for at least 48 hours, at least 72 hours, at least 4 days, at least 5 days, at least 6 days, at least 1 week, at least 2 weeks, at least 3 weeks, at least 4 weeks, at least 6 weeks, at least 8 weeks, at least 12 weeks, or more. Typically this is assessed relative to the last administration of the MEK1/2 inhibitor.

In the context of the therapeutic uses and methods of the invention, a “subject” or “patient” (these terms are used interchangeably herein) is any animal or patient that would benefit from the treatment of HHT-associated telangiectasia, HHT-induced haemorrhage and/or HHT-associated (HHT-induced) anaemia. Typical animal patients are mammals, such as primates, or horses. Preferably the patient is a human.

Thus, the present invention provides a MEK1/2 inhibitor for use in the treatment and/or prevention of telangiectasia associated with HHT, such as nasal telangiectasia, GI telangiectasia, nasopharyngeal telangiectasia, endobronchial telangiectasia and/or tongue telangiectasia, or any combination thereof. Said inhibitor may treat or prevent HHT-associated (HHT-induced) anaemia and/or haemorrhage. Accordingly, the present invention provides a MEK1/2 inhibitor for use in the treatment and/or prevention of HHT-associated (HHT-induced) anaemia and/or HHT-associated (HHT-induced) haemorrhage. The HHT-induced anaemia may be associated with recurrent haemorrhages and/or the HHT induced haemorrhage may be recurrent, as defined herein. In some embodiments, the anaemia and/or haemorrhage, particularly when recurrent, is not responsive to oral iron supplements as described herein. Thus, as described herein, the present invention is particularly useful in treating and/or preventing telangiectasia associated with HHT, HHT-associated (HHT-induced) anaemia, and/or HHT-associated (HHT-induced) haemorrhage which would otherwise require an invasive treatment such as treatment comprising surgery, an anti-angiogenic agent and/or blood transfusion.

The invention also provides a method of treating and/or preventing telangiectasia associated with HHT, HHT-associated (HHT-induced) anaemia, and/or HHT-associated (HHT-induced) haemorrhage comprising administering a therapeutically effective amount of a MEK1/2 inhibitor as defined herein to a patient in need thereof. Preferably said MEK1/2 inhibitor is trametinib.

Additionally, the present invention provides the use of a MEK1/2 inhibitor in the manufacture of a medicament for use in a method of treating and/or preventing telangiectasia associated with HHT, HHT-associated (HHT-induced) anaemia, and/or HHT-associated (HHT-induced) haemorrhage. Preferably said MEK1/2 inhibitor is trametinib.

The therapeutic use or method of the invention may comprise administering a therapeutically effective amount of a MEK1/2 inhibitor of the invention (as defined herein), either alone or in combination with other therapeutic agents, to a patient.

As used herein, the term “treatment” or “treating” embraces therapeutic or preventative/prophylactic measures.

The MEK1/2 inhibitors of the invention may also be used as a preventative therapy. As used herein, the term “preventing” includes preventing the onset of symptoms associated with telangiectasia associated with HHT, HHT-associated (HHT-induced) anaemia, and/or HHT-associated (HHT-induced) haemorrhage (as described herein) and/or reducing the severity or intensity of said symptoms.

A MEK1/2 inhibitor of the invention or a composition comprising a MEK1/2 inhibitor may be administered to a patient already having HHT, HHT-associated (HHT-induced) anaemia, and/or HHT-associated (HHT-induced) haemorrhage, and typically who is exhibiting one or more symptom as described herein. When administered to such a patient, a compound or composition of the invention can cure, delay, reduce the severity of, or ameliorate one or more symptoms, and/or prolong the survival of a patient beyond that expected in the absence of such treatment.

The treatments and preventative therapies of the present invention are applicable to a variety of different patients of different ages. In the context of humans, the therapies are applicable to children (e.g. infants, children under 5 years old, older children or teenagers) and adults, particularly to adults. In the context of other animal subjects (e.g. mammals such as primates), the therapies are applicable to immature subjects and mature/adult subjects.

The MEK1/2 inhibitor of the invention or a composition comprising a MEK1/2 inhibitor may be used in combination with one or more additional therapeutic agents or treatments, which typically may be selected from a conventional treatment for HHT. As a non-limiting example, a MEK1/2 inhibitor of the invention may be used in combination with an oral iron supplement, and/or an agent which protects against haemolysis. When used in combination with one or more additional therapeutic agents or treatments, a MEK1/2 inhibitor of the invention or a composition comprising a MEK1 inhibitor of the invention may be administered before, simultaneously with, or after the administration of the one or more additional therapeutic agent or treatment.

As such, the invention also provide compositions comprising a MEK1/2 inhibitor and one or more additional therapeutic agents (e.g. an oral iron supplement or anti-haemolytic agent) as a combined preparation for simultaneous, separate or sequential use in a method of treating and/or preventing HHT, HHT-associated (HHT-induced) anaemia, and/or HHT-associated (HHT-induced) haemorrhage.

Sequential administration may mean that the two products are administered immediately one after the other, or that the second product is administered within 1 minute, within two minutes, within three minutes, within four minutes, within five minutes, within 10 minutes, within 15 minutes, within 20 minutes, within 25 minutes, within 30 minutes, within 45 minutes, within one hour, or more of the first product being administered.

Separate administration may mean that the second product is administered within one hour, within two hours, within three hours, within six hours, within 12 hours, within 24 hours, within 2 days, within 3 days, within 4 days, within 5 days, within 6 days, within 7 days or more of the first product being administered.

In some embodiments, a MEK1/2 inhibitor of the invention or a composition comprising a MEK1/2 inhibitor is not used as part of a combination therapy. Thus, a MEK1/2 inhibitor of the invention or a composition comprising a MEK1/2 inhibitor may be used independently from any other therapeutic agents or treatments, such as any anti-angiogenic agents. In some embodiments, a MEK1/2 inhibitor of the invention or a composition comprising a MEK1/2 inhibitor is used independently from a PIK3 inhibitor.

### Pharmaceutical Compositions and Formulations

The invention provides a pharmaceutical composition comprising a MEK1/2 inhibitor of the invention and a pharmaceutically acceptable carrier, excipient, diluent, adjuvant, propellant and/or salt. Preferably the MEK1/2 inhibitor is trametinib.

Administration of MEK1/2 inhibitors or compositions of the present invention (e.g. trametinib or a composition comprising trametinib) is generally by conventional routes e.g. oral, intravenous, subcutaneous, intraperitoneal, or mucosal routes. The administration may be by parenteral injection, for example, a subcutaneous, intradermal or intramuscular injection. For example, formulations comprising MEK1/2 inhibitors or compositions of the present invention (e.g. trametinib or a composition comprising trametinib) may be particularly suited to administration orally. Administration of small molecule MEK1/2 inhibitors may be injection, such as intravenously, intramuscularly, intradermally, or subcutaneously, or preferably by oral administration (small molecules with molecule weight of less than 500 Da typically exhibiting oral bioavailability).

Accordingly, MEK1/2 inhibitors or compositions of the present invention (e.g. trametinib or a composition comprising trametinib) may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

Preferably MEK1/2 inhibitors or compositions of the present invention (e.g. trametinib or a composition comprising trametinib) are prepared as for oral administration. A MEK1/2 inhibitor may be encapsulated within an oral dosage form. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

The active ingredients (such as the MEK1/2 inhibitors of the invention) are often mixed with excipients which are pharmaceutically acceptable and compatible with the active ingredient. Suitable excipients are, for example, water, saline, dextrose, glycerol, ethanol, or the like and combinations thereof. In addition, if desired, the composition may contain minor amounts of auxiliary substances such as wetting or emulsifying agents, pH buffering agents, and/or adjuvants which enhance the effectiveness of the MEK1/2 inhibitor.

Generally, the carrier is a pharmaceutically-acceptable carrier. Non-limiting examples of pharmaceutically acceptable carriers include water, saline, and phosphate-buffered saline. In some embodiments, however, where the composition comprises a compound or products of the invention, this may be in lyophilized form, in which case it may include a stabilizer, such as BSA. In some embodiments, it may be desirable to formulate the composition with a preservative, such as thiomersal or sodium azide, to facilitate long term storage.

Examples of additional adjuvants which may be effective include but are not limited to: complete Freunds adjuvant (CFA), Incomplete Freunds adjuvant (IFA), Saponin, a purified extract fraction of Saponin such as Quil A, a derivative of Saponin such as QS-21, lipid particles based on Saponin such as ISCOM/ISCOMATRIX, E. coli heat labile toxin (LT) mutants such as LTK63 and/or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryl oxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, the MF59 formulation developed by Novartis, and the AS02, AS01, AS03 and AS04 adjuvant formulations developed by GSK Biologicals (Rixensart, Belgium).

Examples of buffering agents include, but are not limited to, sodium succinate (pH 6.5), and phosphate buffered saline (PBS; pH 6.5 and 7.5).

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Additional formulations which are suitable for other modes of administration include suppositories and, in some cases, formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

A MEK1/2 inhibitor or composition of the present invention (e.g. trametinib or a composition comprising trametinib) may be administered at daily, weekly, fortnightly or monthly intervals. Typically a MEK1/2 inhibitor or composition of the present invention (e.g. trametinib or a composition comprising trametinib) is for daily administration.

The dosage ranges for administration of the MEK1/2 inhibitors or compositions of the present invention (e.g. trametinib or a composition comprising trametinib) are those to produce the desired therapeutic effect. It will be appreciated that the dosage range required depends on the precise nature of the MEK1/2 inhibitor or composition, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

In some embodiments, the dose of MEK1/2 inhibitor, particularly trametinib is lower than the standard dose of said MEK1/2 inhibitor used as a chemotherapeutic. By way of non-limiting example, a MEK1/2 inhibitor (e.g. trametinib) may be used according to the invention at a dose of less than 2 mg/day (2 mg qd), less than 1.5 mg/day (1.5 mg qd), less than 1 mg/day (1 mg qd), or less than 0.5 mg/day (0.5 mg qd). Lower doses may be preferred to reduce the peak plasma concentration and/or elimination half-life of the MEK1/2 inhibitor, since a trametinib dose of 2 mg/day results in a peak plasma concentration (Cmax) of 22.2 ng/ml, and elimination half-life of 3.9-4.8 days.

In some embodiments, the frequency of MEK1/2 inhibitor, particularly trametinib is lower than the standard frequency of said MEK1/2 inhibitor used as a chemotherapeutic. By way of non-limiting example, a MEK1/2 inhibitor (e.g. trametinib) may be used according to the invention at a daily dose, alternate daily dose or three times weekly dose. Again, lower doses may be preferred to reduce the peak plasma concentration and/or elimination half-life of the MEK1/2 inhibitor, since a dose of 2 mg/day results in a peak plasma concentration (Cmax) of 22.2 ng/ml, and elimination half-life of 3.9-4.8 days.

The MEK1/2 inhibitors or compositions of the present invention (e.g. trametinib or a composition comprising trametinib) may be administered for less than 8 weeks, for 8-52 weeks; for example, 12-48 weeks, 16-44 weeks, 20-40 weeks, or 24-36 weeks or more as a chronic treatment, preferably for the life of the patient.

The duration of action of a MEK1/2 inhibitor according to the invention may be for at least 48 hours, at least 72 hours, at least 4 days, at least 5 days, at least 6 days, at least 1 week, at least 2 weeks, at least 3 weeks, at least 4 weeks, at least 6 weeks, at least 8 weeks, at least 12 weeks, or more. Typically this is assessed relative to the last administration of the MEK1/2 inhibitor.

The MEK1/2 inhibitors or compositions of the present invention (e.g. trametinib or a composition comprising trametinib) may be administered in a manner that prolongs the duration of the bioavailability of the MEK1/2 inhibitor, increases the duration of action of the MEK1/2 inhibitor and the release time frame of the MEK1/2 inhibitor by an amount selected from the group consisting of at least 12 hours, at least 24 hours, at least 48 hours, at least 72 hours, at least 4 days, at least 5 days, at least 6 days, at least 7 days, at least 2 weeks, at least 3 weeks, and at least a month, over that of the MEK1/2 inhibitor in the absence of the duration-extending administration. Optionally, the duration of any or all of the preceding effects is extended by at least 12 hours, at least 24 hours, at least 48 hours, at least 72 hours, at least 4 days, at least 5 days, at least 6 days, at least 7 days, at least 2 weeks, at least 3 weeks or at least a month.

The invention provides compositions comprising a MEK1/2 inhibitor (as described herein) and one or more additional therapeutic agents or treatments, Said one or more additional therapeutic agent or treatment may typically be selected from a conventional treatment for HHT. A composition of the invention may comprise a MEK1/2 inhibitor of the invention and an agent which supports red blood cell synthesis, and/or an agent which protects against haemolysis. As a non-limiting example, a composition of the invention may comprise a MEK1/2 inhibitor and one or more of a B group vitamin (such as thiamine and/B12), folate, L-thyroxine, or an antibiotic.

### Screening

The invention also provides a method of identifying a patient as suitable for use of a MEK1/2 inhibitor for treating and/or preventing telangiectasia associated with HHT, HHT-associated haemorrhage and/or HHT-associated anaemia, as described herein.

Identifying a patient as suitable for a treatment of the invention may also be determined by assessing clinical indicators of haemorrhage and/or anaemia. For example, a clinician's categorisation of a patient's clinical symptoms (using standard clinical classification/categorisation criteria) may be used to determine whether a patient is suitable for treatment. It is within the routine skill of a clinician to use standard techniques, such as those described herein, to identify patients suitable for treatment according to the present invention based on cut-off/threshold values or other clinically-relevant criteria.

By way of non-limiting example, a patient may be identified on the basis of one or more of: (i) red cell indices; (ii) impaired activity; (iii) lack of responsiveness to dietary or oral iron supplements; and/or (iv) lack of tolerance of patients to bleeding and/or anaemia.

Red cell indices which may be used to identify a patient as suitable for treatment according to the invention include. The lower limit of a normal haemoglobin is considered about 115 g/L (11.5 g/dL), lower limit of haematocrit as about 0.35, lower limit of mean corpuscular volume (MCV) as about 83.5 fL, lower limit of mean corpuscular haemoglobin (MCH) as about 27.5 pg, and lower limit of mean corpuscular haemoglobin concentration (MCHC) as about 315 g/L (31.5 g/dL). In some embodiments, the combination of any two values below the lower limit of normal range despite tolerable doses of oral iron treatment may be used to identify a patient as suitable for treatment. These indices may also be associated with evidence of any one or more iron indices below the lower limit of the normal range which is usually considered as about 10 μmol/L for iron, about 20% for transferrin saturation index and about 20 μg/L for ferritin.

Alternatively or in addition, arterial oxygen content values may be used to identify a patient as suitable for treatment according to the invention. Arterial oxygen content values which may be used to identify a patient as suitable for treatment according to the invention include provision for patients who need a higher than normal haemoglobin to maintain arterial oxygen content (CaO₂) due to low oxygen saturation (SaO₂) of haemoglobin in arterial blood, either because of pathology such as HHT-associated pulmonary AVMs, or due to barometric falls in partial pressure of oxygen at altitude. In both settings, higher red cell counts, haemoglobin and haematocrit is required to maintain a normal CaO₂ where the median value for non-iron deficient individuals with HHT was 18.8 mls of oxygen per dL, and CaO₂ calculated by [haemoglobin]×[SaO₂]×1.34 with 1.34 representing the mls of oxygen carried by one gram of haemoglobin. For an individual or patient with a normal arterial SaO₂ of 97%, a normal CaO₂ can be delivered with a normal haemoglobin of 12.3 g/dL. For an individual with a low arterial SaO₂, a normal CaO₂ can only be delivered by a haemoglobin above the upper limit of normal which is quoted as 15 g/dL (150 g/L). For an individual with an arterial SaO₂ of 93% (the median value for a pulmonary AVM patient on presentation), a normal CaO₂ requires a haemoglobin of 15.1 g/dL (151 g/L), for an individual with an SaO₂ of 80%, a normal CaO₂ requires a haemoglobin of 17.5 g/dL (175 g/L), and for an individual with an SaO₂ of 65% (the lowest steady state in our cohort), a normal CaO₂ requires a haemoglobin of 21.6 g/dL (216 g/L).

Impaired activity criteria which may be used to identify a patient as suitable for treatment according to the invention include a patient being allocated with “impaired exercise tolerance” using a standard Qol questionnaire, such as the Veterans Specific Activity Questionnaire (VSAQ) (described in Gawecki et al. BMJ Open Resp. Res. (2019) 6: e000351, PMID 30956797, which is herein incorporated by reference in its entirety). A patient may be identified as suitable for treatment according to the invention if their VSAQ score is at least 2 points lower than their VSAQ score when not anaemic, or compared with the VSAQ score of a matched (e.g. age, sex, and prior exercise-matched) healthy control.

Lack of responsiveness or failure to tolerate dietary or oral iron supplements may be used to identify a patient as suitable for treatment according to the invention include those described herein. Thus, a patient may be identified as suitable for treatment according to the invention if they are not responsive to dietary iron and/or oral iron supplements in the range of from about 25 mg to about 200 mg, such as from about 35 mg to 100 mg/day (e.g. a treatment regimen of a 35 mg or 65 mg oral dose 1-2 times per day), noting very few HHT patients are able to tolerate the “usual” treatment regime of 195 mg (e.g. a treatment regimen of a 65 mg oral dose 3 times per day).

Lack of tolerance of patients to bleeding and/or anaemia criteria which may be used to identify a patient as suitable for treatment according to the invention include a patient identifying as having an unacceptable quality of life using a standard Qol questionnaire (such as those described herein). Other criteria include a patient not being amenable to local treatments because of the specific features or sites of telangiectasia, e.g. those near the back of the nares; on the tongue or lips; and in the nasopharynx or endobronchial tree.

Accordingly, the invention provides a method of identifying a patient as suitable for use of a MEK1/2 inhibitor for treating and/or preventing telangiectasia associated with HHT, HHT-associated haemorrhage and/or HHT-associated anaemia, as described herein, which comprises determining and/or quantifying one or more of a patient's (i) red cell indices; (ii) impaired activity; (iii) lack responsiveness to dietary or oral iron supplements; and/or (iv) lack of tolerance to bleeding and/or anaemia.

### Kits

The invention further provides a kit comprising a MEK1/2 inhibitor or composition of the present invention (e.g. trametinib or a composition comprising trametinib).

Said kit may further comprise one or more additional therapeutic agent or treatment, as described herein.

Components of a kit are generally sterile and in sealed vials or other containers. Kits may be employed in therapy, diagnostic analysis or other applications as described herein.

A kit may contain instructions for use of the components, e.g., for a treatment or method in accordance with the present invention. Ancillary materials to assist in or to enable performing such a treatment or method may be included within a kit of the invention.

Each component of the kits is generally in its own suitable container. Thus, these kits generally comprise distinct containers suitable for each component (each binding member present). Further, the kits may comprise instructions for performing the treatment or method, and/or for interpreting and analysing data resulting from the performance of the treatment or method.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264 (4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8 (5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M-A New Algorithm for Multiple Alignment of Highly Divergent Sequences, 20 (9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

A R N D C Q E G H I L K M F P S T W Y V

A 4
R −1 5
N −2 0 6
D −2 −2 1 6
C 0 −3 −3 −3 9
Q −1 1 0 0 −3 5
E −1 0 0 2 −4 2 5
G 0 −2 0 −1 −3 −2 −2 6
H −2 0 1 −1 −3 0 0 −2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5
F −2 −3 −3 −3 −2 −3 −3 −3 −1 0 0 −3 0 6
P −1 −2 −2 −1 −3 −1 −1 −2 −2 −3 −3 −1 −2 −4 7
S 1 −1 1 0 −1 0 0 0 −1 −2 −2 0 −1 −2 −1 4
T 0 −1 0 −1 −1 −1 −1 −2 −2 −1 −1 −1 −1 −2 −1 1 5
W −3 −3 −4 −4 −2 −2 −3 −2 −2 −3 −2 −3 −1 1 −4 −3 −2 1 1
Y −2 −2 −2 −3 −2 −1 −2 −3 2 −1 −1 −2 −1 3 −3 −2 −2 2 7
V 0 −3 −3 −3 −1 −2 −2 −3 −3 3 1 −2 1 −1 −2 −2 0 −3 −1 4

The percent identity is then calculated as:

$$\frac{Total number of identical matches}{[ length of the longer sequence plus the number of gaps introduced into the longer sequence in order to align the two sequences ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
glycine Small:
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### Sequence Information

Where an initial Met amino acid residue or a corresponding initial codon is indicated in any of the following SEQ ID NOs, said residue/codon is optional.

-human MEK1 nucleic acid sequence of NCBI Accession No. NM_002755
(version 4, i.e. NM_002755.4, accessed 22 Jan. 2022)
SEQ ID NO: 1
agtccctccc agggccgctt cgcagagcgg ctaggagcac ggcggcggcg gcactttccc cggcaggagc
tggagctggg ctctggtgcg cgcgcggctg tgccgcccga gccggaggga ctggttggtt gagagagaga
gaggaaggga atcccgggct gccgaaccgc acgttcagcc cgctccgctc ctgcagggca gcctttcggc
tctctgcgcg cgaagccgag tcccgggcgg gtggggcggg ggtccactga gaccgctacc ggcccctcgg
cgctgacggg accgcgcggg gcgcacccgc tgaaggcagc cccggggccc gcggcccgga cttggtcctg
cgcagcgggc gcggggcagc gcagcgggag gaagcgagag gtgctgccct ccccccggag ttggaagcgc
gttacccggg tccaaaatgc ccaagaagaa gccgacgccc atccagctga acccggcccc cgacggctct
gcagttaacg ggaccagctc tgcggagacc aacttggagg ccttgcagaa gaagctggag gagctagagc
ttgatgagca gcagcgaaag cgccttgagg cctttcttac ccagaagcag aaggtgggag aactgaagga
tgacgacttt gagaagatca gtgagctggg ggctggcaat ggcggtgtgg tgttcaaggt ctcccacaag
ccttctggcc tggtcatggc cagaaagcta attcatctgg agatcaaacc cgcaatccgg aaccagatca
taagggagct gcaggttctg catgagtgca actctccgta catcgtgggc ttctatggtg cgttctacag
cgatggcgag atcagtatct gcatggagca catggatgga ggttctctgg atcaagtcct gaagaaagct
ggaagaattc ctgaacaaat tttaggaaaa gttagcattg ctgtaataaa aggcctgaca tatctgaggg
agaagcacaa gatcatgcac agagatgtca agccctccaa catcctagtc aactcccgtg gggagatcaa
gctctgtgac tttggggtca gcgggcagct catcgactcc atggccaact ccttcgtggg cacaaggtcc
tacatgtcgc cagaaagact ccaggggact cattactctg tgcagtcaga catctggagc atgggactgt
ctctggtaga gatggcggtt gggaggtatc ccatccctcc tccagatgcc aaggagctgg agctgatgtt
tgggtgccag gtggaaggag atgcggctga gaccccaccc aggccaagga cccccgggag gccccttagc
tcatacggaa tggacagccg acctcccatg gcaatttttg agttgttgga ttacatagtc aacgagcctc
ctccaaaact gcccagtgga gtgttcagtc tggaatttca agattttgtg aataaatgct taataaaaaa
ccccgcagag agagcagatt tgaagcaact catggttcat gcttttatca agagatctga tgctgaggaa
gtggattttg caggttggct ctgctccacc atcggcctta accagcccag cacaccaacc catgctgctg
gcgtctaagt gtttgggaag caacaaagag cgagtcccct gcccggtggt ttgccatgtc gcttttgggc
ctccttccca tgcctgtctc tgttcagatg tgcatttcac ctgtgacaaa ggatgaagaa cacagcatgt
gccaagattc tactcttgtc atttttaata ttactgtctt tattcttatt actattattg ttcccctaag
tggattggct ttgtgcttgg ggctatttgt gtgtatgctg atgatcaaaa cctgtgccag gctgaattac
agtgaaattt tggtgaatgt gggtagtcat tcttacaatt gcactgctgt tcctgctcca tgactggctg
tctgcctgta ttttcgggat tctttgacat ttggtggtac tttattcttg ctgggcatac tttctctcta
ggagggagcc ttgtgagatc cttcacaggc agtgcatgtg aagcatgctt tgctgctatg aaaatgagca
tcagagagtg tacatcatgt tattttatta ttattatttg cttttcatgt agaactcagc agttgacatc
caaatctagc cagagccctt cactgccatg atagctgggg cttcaccagt ctgtctactg tggtgatctg
tagacttctg gttgtatttc tatatttatt ttcagtatac tgtgtgggat acttagtggt atgtctcttt
aagttttgat taatgtttct taaatggaat tattttgaat gtcacaaatt gatcaagata ttaaaatgtc
ggatttatct ttccccatat ccaagtacca atgctgttgt aaacaacgtg tatagtgcct aaaattgtat
gaaaatcctt ttaaccattt taacctagat gtttaacaaa tctaatctct tattctaata aatatactat
gaaataaaaa aaaaaggatg aaagcta
-human MEK1 protein sequence of UniProt Accession No. Q02750
(sequence version 2, accessed 22 Jan. 2022)
SEQ ID NO: 2
MPKKKPTPIQ LNPAPDGSAV NGTSSAETNL EALQKKLEEL ELDEQQRKRL EAFLTQKQKV GELKDDDFEK
ISELGAGNGG VVFKVSHKPS GLVMARKLIH LEIKPAIRNQ IIRELQVLHE CNSPYIVGFY GAFYSDGEIS
ICMEHMDGGS LDQVLKKAGR IPEQILGKVS IAVIKGLTYL REKHKIMHRD VKPSNILVNS RGEIKLCDFG
VSGQLIDSMA NSFVGTRSYM SPERLQGTHY SVQSDIWSMG LSLVEMAVGR YPIPPPDAKE LELMFGCQVE
GDAAETPPRP RTPGRPLSSY GMDSRPPMAI FELLDYIVNE PPPKLPSGVF SLEFQDFVNK CLIKNPAERA
DLKQLMVHAF IKRSDAEEVD FAGWLCSTIG LNQPSTPTHA AGV
-human MEK2 nucleic acid sequence of NCBI Accession No. NM_030662
(version 4, i.e. NM_030662.4, accessed 22 Jan. 2022)
SEQ ID NO: 3
ctctcggact cgggctgcgg cgtcagcctt cttcgggcct cggcagcggt agcggctcgc tcgcctcagc
cccagcgccc ctcggctacc ctcggcccag gcccgcagcg ccgcccgccc tcggccgccc cgacgccggc
ctgggccgcg gccgcagccc cgggctcgcg taggcgccga ccgctcccgg cccgccccct atgggccccg
gctagaggcg ccgccgccgc cggcccgcgg agccccgatg ctggcccgga ggaagccggt gctgccggcg
ctcaccatca accctaccat cgccgagggc ccatccccta ccagcgaggg cgcctccgag gcaaacctgg
tggacctgca gaagaagctg gaggagctgg aacttgacga gcagcagaag aagcggctgg aagcctttct
cacccagaaa gccaaggtcg gcgaactcaa agacgatgac ttcgaaagga tctcagagct gggcgcgggc
aacggcgggg tggtcaccaa agtccagcac agaccctcgg gcctcatcat ggccaggaag ctgatccacc
ttgagatcaa gccggccatc cggaaccaga tcatccgcga gctgcaggtc ctgcacgaat gcaactcgcc
gtacatcgtg ggcttctacg gggccttcta cagtgacggg gagatcagca tttgcatgga acacatggac
ggcggctccc tggaccaggt gctgaaagag gccaagagga ttcccgagga gatcctgggg aaagtcagca
tcgcggttct ccggggcttg gcgtacctcc gagagaagca ccagatcatg caccgagatg tgaagccctc
caacatcctc gtgaactcta gaggggagat caagctgtgt gacttcgggg tgagcggcca gctcatcgac
tccatggcca actccttcgt gggcacgcgc tcctacatgg ctccggagcg gttgcagggc acacattact
cggtgcagtc ggacatctgg agcatgggcc tgtccctggt ggagctggcc gtcggaaggt accccatccc
cccgcccgac gccaaagagc tggaggccat ctttggccgg cccgtggtcg acggggaaga aggagagcct
cacagcatct cgcctcggcc gaggcccccc gggcgccccg tcagcggtca cgggatggat agccggcctg
ccatggccat ctttgaactc ctggactata ttgtgaacga gccacctcct aagctgccca acggtgtgtt
cacccccgac ttccaggagt ttgtcaataa atgcctcatc aagaacccag cggagcgggc ggacctgaag
atgctcacaa accacacctt catcaagcgg tccgaggtgg aagaagtgga ttttgccggc tggttgtgta
aaaccctgcg gctgaaccag cccggcacac ccacgcgcac cgccgtgtga cagtggccgg gctccctgcg
tcccgctggt gacctgccca ccgtccctgt ccatgccccg cccttccagc tgaggacagg ctggcgcctc
cacccaccct cctgcctcac ccctgcggag agcaccgtgg cggggcgaca gcgcatgcag gaacgggggt
ctcctctcct gcccgtcctg gccggggtgc ctctggggac gggcgacgct gctgtgtgtg gtctcagagg
ctctgcttcc ttaggttaca aaacaaaaca gggagagaaa aagcaaa
-human MEK2 protein sequence of UniProt Accession No. P36507
(sequence, accessed 22 Jan. 2022)
SEQ ID NO: 4
MLARRKPVLP ALTINPTIAE GPSPTSEGAS EANLVDLQKK LEELELDEQQ KKRLEAFLTQ KAKVGELKDD
DFERISELGA GNGGVVTKVQ HRPSGLIMAR KLIHLEIKPA IRNQIIRELQ VLHECNSPYI VGFYGAFYSD
GEISICMEHM DGGSLDQVLK EAKRIPEEIL GKVSIAVLRG LAYLREKHQI MHRDVKPSNI LVNSRGEIKL
CDFGVSGQLI DSMANSFVGT RSYMAPERLQ GTHYSVQSDI WSMGLSLVEL AVGRYPIPPP DAKELEAIFG
RPVVDGEEGE PHSISPRPRP PGRPVSGHGM DSRPAMAIFE LLDYIVNEPP PKLPNGVFTP DFQEFVNKCL
IKNPAERADL KMLTNHTFIK RSEVEEVDFA GWLCKTLRLN QPGTPTRTAV

### EXAMPLES

The invention will be further clarified by the following examples, which are intended to be purely exemplary of the invention and are in no way limiting.

### Example 1—Trametinib Effectively Treated Haemorrhage and Anaemia in HHT Patient

A patient over the age of 60 was suffering with both HHT and a progressive cancer. The patient's HHT was caused by a loss-of-function variant (“mutation”) in the ENG gene encoding endoglin. The patient had been under review by the HHT services at Imperial College by the inventor for over a decade. Key features of the patient's HHT included nosebleeds and transfusion-dependent anaemia, together with pulmonary arteriovenous malformations (PAVMs).

The oncologist responsible for the patient's cancer treatment initiated chemotherapy with trametinib according to a standard chemotherapeutic regimen. The patient reported only minor side effects corresponding to Grade 1 adverse events using the common terminology criteria for adverse events (CTCAE, version 5.0, Nov. 27, 2017, NIH 2017).

Prior to commencing treatment with trametinib, the patient had nosebleeds that varied between several times per week and multiple times per day, requiring specialist ENT treatments. In this respect, the patient was similar to ˜50% of adult HHT populations, and thus exemplifies the HHT cohort.

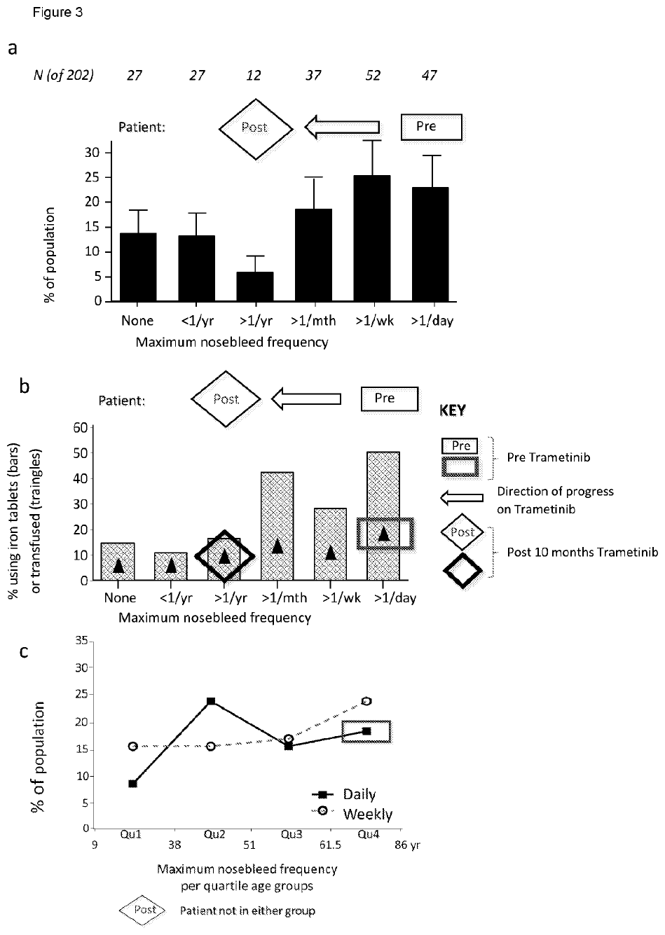

The patient was reviewed at regular intervals following initiation of trametinib. When reviewed by the HHT service after 10 months trametinib treatment, the patient spontaneously volunteered that since starting trametinib, nosebleeds had reduced in frequency to only from “time to time”. When questioned to determine frequency grouping, they suggested no more than once per month, corresponding to the 3rd lowest frequency grade of nosebleeds for HHT patients (see FIGS. 3A and B). In addition, the intensity of the patient's nosebleeds had reduced to “brief trickles”.

Prior to commencing trametinib, the patient had been receiving regular blood transfusions for many years for anaemia. In this respect, the patient was similar to ˜5-10% of adult HHT populations.

When reviewed after 10 months trametinib treatment, the patient stated that they had previously required blood (red cell) transfusions every 3 weeks for anaemia but since starting trametinib, far fewer were needed: Assessments had been extended to 8 weekly, and transfusions were not required even at extended assessment times (as shown in FIGS. 4A and B).

Based on this assessment, a clinically important improvement in nosebleeds and anaemia resulted from trametinib treatment.

The patient persisted with trametinib treatment, with treatment duration at time of filing being over 2 years' continuous treatment. At 18 and 24 months, the improvements in nosebleeds (frequency and intensity) were maintained at approximately one a year, with a reduced haemorrhage adjusted iron requirement (HAIR) (not shown).

Example 2-Trametinib had no effect on the PAVMs of the HHT patient The effect of trametinib treatment on the patient's AVMs was assessed in parallel. Thoracic CT scan images taken for clinical purposes were compared before Trametinib (−10 years (T−10 yrs), −2 years (T−2ys), −1 month (T−1M)), and after Trametinib for 4 months (T+4M), 10 months (T+10M) and 18 months (T+18M, data not shown).

Blinded analysis of the CT scans was conducted by an expert Imperial Thoracic Radiologist to address the question of “whether the PAVMs have stayed the same, got better, or got worse?”

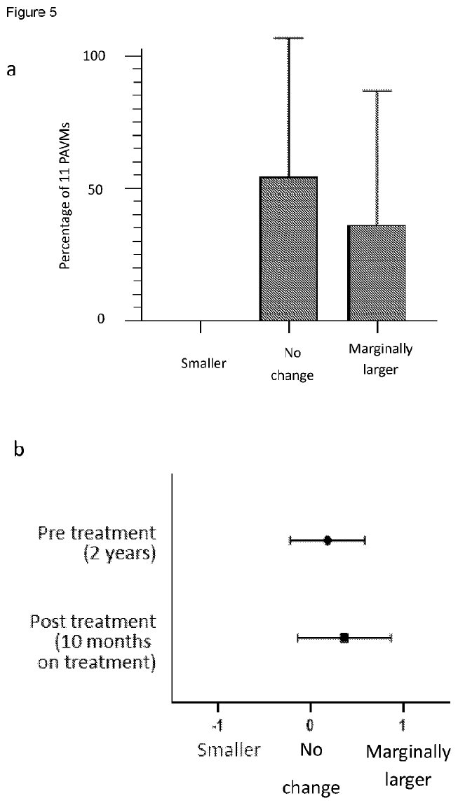

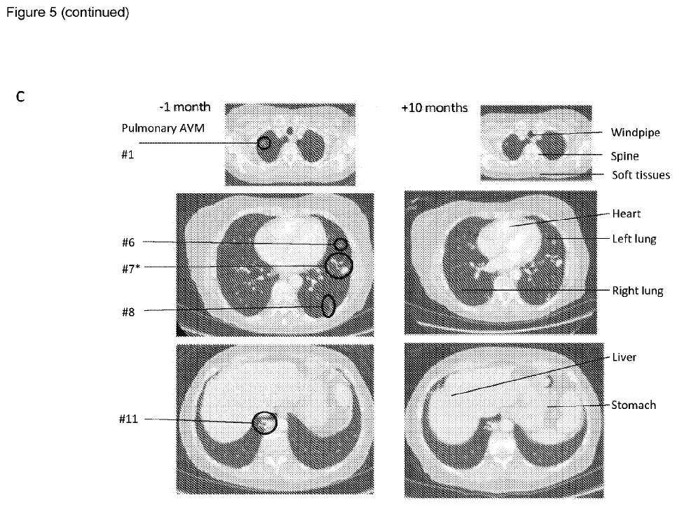

The blinded analyser concluded that the venous sacs of some of the larger PAVMs have increased in size very slightly between T−2 ys to T−1M. The changes (between T−1M and T+10M) were within the error of CT, but with a probable slight increase in size.” A specific venous sac (#7) was measured at 13 mm (T−2 ys), 15 mm (T−1M), 16 mm (T+4M), 16.6 mm (T+10M). Annotated CT scans are shown in FIG. 5C. As quantified in FIGS. 5A and B, no PVAMs improved during the treatment, and some were slightly larger.

Therefore, whilst treatment with trametinib elicited an improvement in HHT-associated haemorrhage (nosebleeds) and anaemia, pulmonary AVMs did not improve with trametinib treatment, and their natural history was not observably altered.

### Example 3—Cellular Background: Canonical (SMAD-Dependent) Signalling Appears Less Affected in HHT BOECs than Non-Canonical (SMAD-Independent) Pathways

TGFβ and BMP9 signalling are essential for development and viability. It has previously been reported that endoglin heterozygous mouse and human endothelial cells compensate by lowering expression of the ALK1 receptor encoded by ACVRL1; that ACVRL1 heterozygous human endothelial cells compensate by lowering expression of endoglin, but that intracellular signal transduction pathways below these receptors diverge. TGF-β and BMP ligands can signal through the canonical (SMAD4) pathway which includes 3 components encoded by HHT genes ACVRL1, ENG and SMAD4. There are also separate, SMAD4-independent signal transduction pathways which still rely on primary receptor binding by TGF-β and BMP ligands, and are thus also include components encoded by HHT genes ACVRL1 and ENG. HHT pathogenic variants have been reported to impede both canonical (SMAD4) and non-canonical (non-SMAD4) TGFβ/BMP9 signalling. However, even the effect of HHT pathogenic variants on steady-state gene expression is not as predictable as has been suggested in the art.

The inventors hypothesised that when signalling through the canonical pathway is reduced because of heterozygous loss of ACVRL1, ENG or SMAD4, then in order to survive, cells compensatory mechanisms will be more effective than for the less specific non-canonical signalling pathways. This predicted that HHT endothelial cells heterozygous for ACVRL1 or ENG loss-of-function variants would display more normal responses of target genes for the canonical pathway, than for non-canonical pathways.

To test this hypothesis, further RNASeq analysis of blood outgrowth endothelial cells (BOECs) from HHT patients was carried out. It has previously been shown that the Shovlin group established BOECs from controls and patients of all 3 HHT genotypes (ENG^{+/-}, ACVRL1^{+/-} and SMAD4^{+/-}); extracted RNA from replicate cultures; and developed novel methodologies to interpret primary alignment data generated by sequencing alignments to Homo sapiens human genome build 38 (GRCh38). None of the subsequent data are in the public domain.

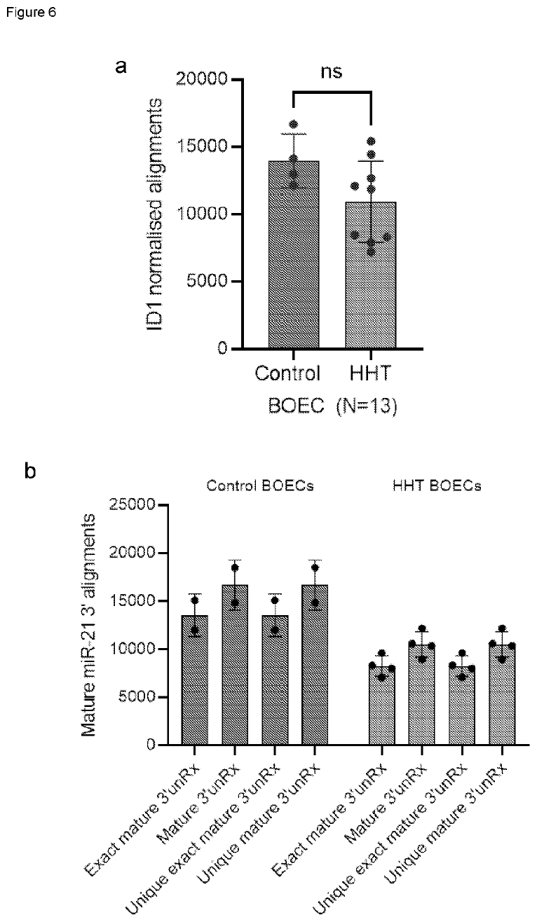

Normally, BMPs increase expression of ID1 by canonical pathways, and promote post-transcriptional processing of primary miR-21 transcript (pri-miR-21) into precursor miR-21 (pre-miR-21) by non-canonical pathways. The mean alignments to ID1 were 13,958 (SD 1,986) in control BOECs and 10,907 (SD 3,037) in ACVRL1^{+/-} and ENG^{+/-} BOECs, i.e. the HHT BOEC alignments were reduced by 22% compared to control alignments for this canonical pathway (FIG. 6A). The mean unique exact alignments to mature 3′ miR-21 were 13,520 (standard deviation [SD] 2,194) in control BOECs and 8,232 (SD 1061) in ACVRL1^{+/-} and ENG^{+/-} BOECs, i.e. the HHT BOEC alignments were reduced by 39% compared control alignments for this non-canonical pathway, with similar patterns exhibited for alignments calculated in slightly less specific ways (FIG. 6B).

Taken together, these evaluations supported the possibility that the canonical signalling pathway was employing compensations downstream of the receptors.

### Example 4—Compensation of Steady-State, Canonical TGFβ/BMP9 Signalling in HHT BOECs by Down-Regulation of TGFβ/BMP9 Signalling Inhibitors

The inventors hypothesised that compensatory mechanisms for the canonical pathway would include down regulation of TGFβ/BMP9 pathway inhibitors to “dampen the brakes”.

To test this hypothesis, further RNAseq analysis of blood outgrowth endothelial cells (BOECs) from HHT patients was carried out, and the relative expression levels of TGFβ/BMP9 signalling inhibitors was investigated. For this, the RNASeq data from the Shovlin group's blood outgrowth endothelial cells (BOECs) from controls and patients of all 3 HHT genotypes (ENG^{+/-}, ACVRL1^{+/}− and SMAD4^{+/-}) was examined further. RNA had been extracted from replicate cultures; sequenced and aligned to Homo sapiens human genome build 38 (GRCh38), and unique gene reads normalised to total alignment counts per library initially further normalised using the Shovlin-group validated, lowest Gini Coefficient (GC) housekeeper gene RBM45

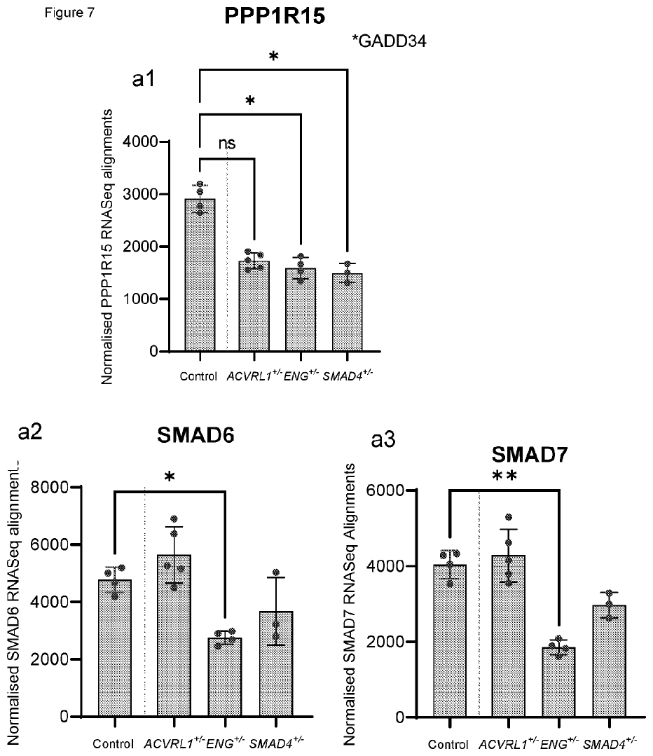

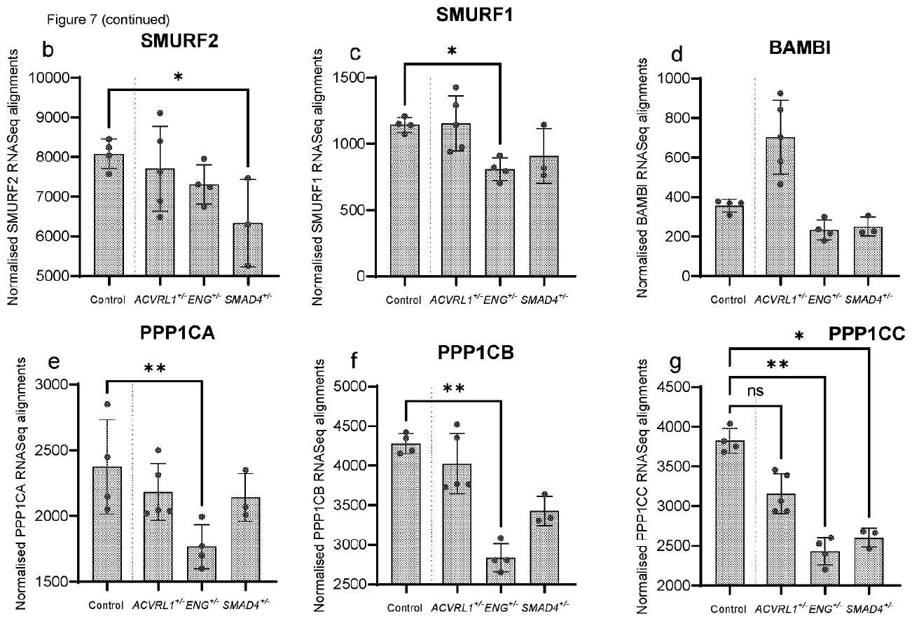

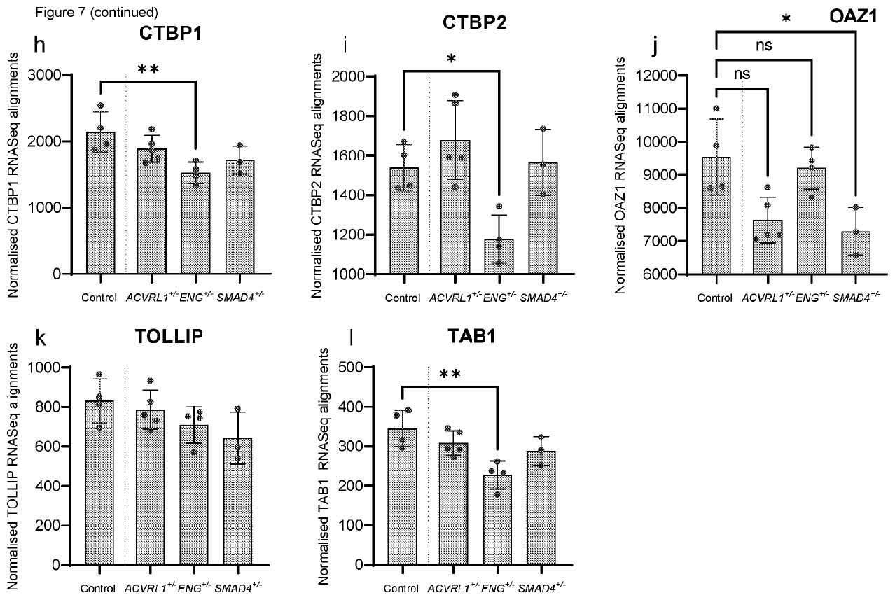

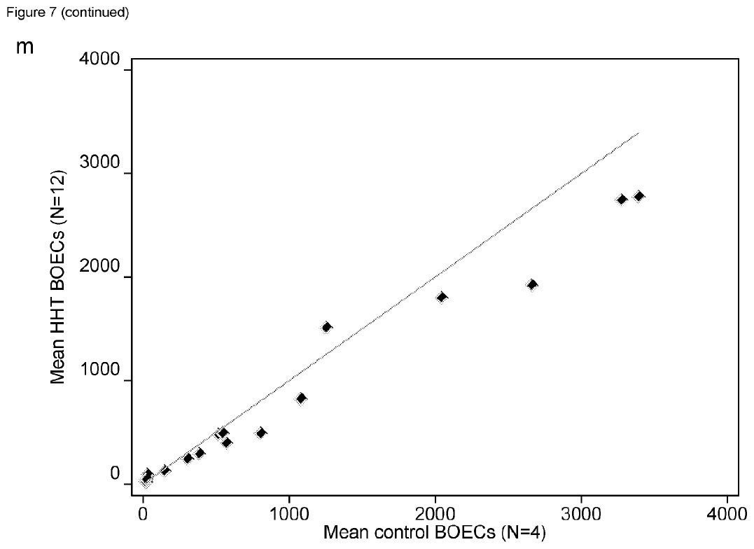

The data in FIG. 7 are of RBM45-normalised alignments to the genes for all moieties defined in Miyazawa and Miyazono (Perspect Biol. 2017, 9 (3): a022095) except for SIK1, which is not considered a pathway inhibitor according to the technical consensus in 2022.

Transcription of SMAD6 and SMAD7 was of particular interest, as these are the best-known of the canonical pathway inhibitors. Expression of SMAD6 and SMAD7 is upregulated by SMAD4 to provide negative feedback loops: SMAD6 preferentially inhibits SMAD signalling initiated by the BMP type 1 receptors ALK-3 and ALK-6, whereas SMAD7 inhibits both BMP9 and TGFβ-induced SMAD signalling.

As shown in FIG. 7, various inhibitors of TGFβ/BMP9 signalling, including SMAD6, SMAD7, SMURFs, CTBPs, TAB and OAF were down-regulated to different extents in BOECs with different HHT pathogenic variants:

FIG. 7a1: PPP1R15 encoding GADD34: reduced in all HHT BOECs

FIG. 7a2: SMAD6: greatest reduction seen in ENG+/− BOECs

FIG. 7a3: SMAD7: greatest reduction seen in ENG+/− BOECs

FIG. 7b: SMURF1: greatest reduction seen in ENG+/− BOECs

FIG. 7c: SMURF2: greatest reduction seen in SMAD4+/− BOECs

FIG. 7d: BAMBI: greatest reduction seen in ENG+/− and ACVRL1+/− BOECs

FIG. 7e-g: PPP1CA, PPP1CB, and PPP1CC encoding protein phosphatase 1 catalytic subunits alpha, beta, gamma: reduced most in ENG+/− BOECs

FIG. 7h: CTBP1: greatest reduction seen in ENG+/− BOECs

FIG. 7i: CTBP2: greatest reduction seen in ENG+/− BOECs

FIG. 7j: OAZ1: greatest reduction seen in SMAD4+/− BOECs

FIG. 7k: TOLLIP: observable reduction seen in ENG+/− and ACVRL1+/− BOECs

FIG. 7l: TAB1: greatest reduction seen in ENG+/− BOECs

Taken together, the data provide strong evidence that HHT BOECs reduce inhibitors of the pathway affected by the HHT-causal variants, most likely as a compensatory mechanism in order to sustain essential signalling levels through SMAD4. In particular, reduction of SMAD6 and SMAD7 minimises SMAD signalling inhibition by these molecules, and so helps to compensate for reduced SMAD4 signalling resulting from the pathogenic SMAD4 or ENG variants.

RNASeq data of the HHT BOECs compared with control BOECs, provided evidence that transcript levels of MAP3K1 (encoding MEKK1) were higher in HHT BOECs compared with the control BOECs, whereas the transcript levels of the other 14 MAP3K genes were similar or lower in HHT BOECs compared with the control BOECs (FIG. 7m).

### Example 5—Compensation of Steady-State TGFβ/BMP9 Signalling in HHT BOECs by Up-Regulation of SMAD4 Signalling Activators

Following on from the results in Example 4, which suggest a compensatory mechanism in the HHT BOECs, the ability and specific mechanism by which HHT cells compensate for defects in TGFβ/BMP9 signalling was further investigated.

In addition to downregulation of in TGFβ/BMP9 signalling inhibitors as demonstrated in Example 4, the inventors further hypothesised that the compensatory mechanisms in HHT BOECs would include upregulation of MEKK1 (encoded by MAP3K1), as MEKK1 has previously been shown to selectively phosphorylate SMAD2 and increase SMAD4 transcription in endothelial cells independently to TGFβ1.

RNASeq was carried out on BOECs from HHT patients and healthy controls as described above. Having recognised that RBM45 was not as invariant in the BOECs as expected, the least variant low GC genes in BOECs under the experimental conditions (HNRNPK, COX411, IK, UBR2, UBE2Q1, SNW1, TXNL1, and KAT5) were used for DeSeq2 normalised expression. It is now evident that the RBM45-normalisations effectively normalise for PTC-related stress, whereas the DeSeq2-normalisations do not.

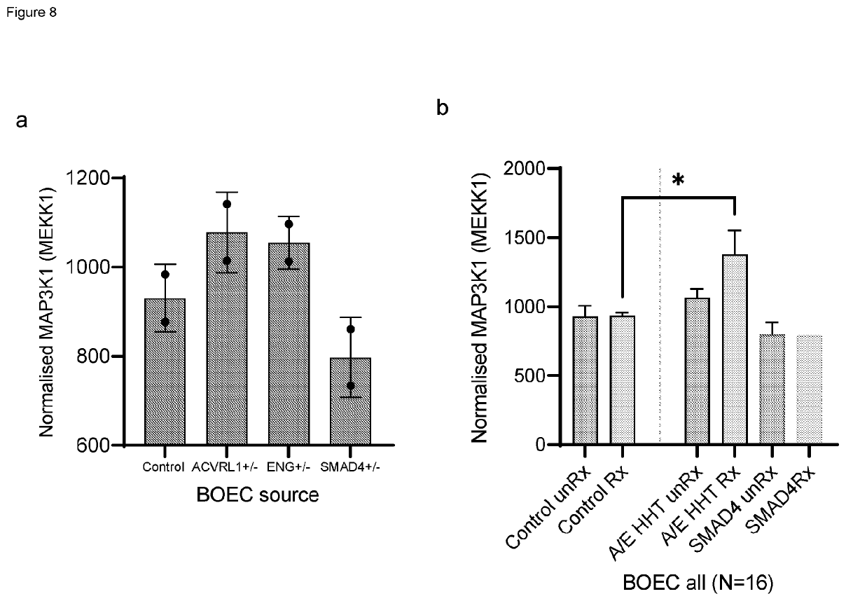

As shown in FIG. 8A irrespective of normalisation-method employed, MAP3K1 transcripts were observably increased in BOECs from HHT patients, particularly those with the ACVRL1^{+/-} or ENG^{+/-} pathogenic variants. Furthermore, MAP3K1 transcripts were significantly increased on BMP9 stimulation (as shown in FIG. 8B).

Upregulation of MAP3K1 stimulates SMAD4 independent of TGFβ/BMP9 signalling. These data therefore implicate upregulation of MAP3K1 as a compensatory response in HHT BOECs.

However, MAP3K1 has additional effects other than stimulating SMAD4 signalling. Therefore, as a next step the inventors investigated the effect of increased MAP3K1 signalling on other pathways.

### Example 6—Up-Regulation of MAP3K1 Upregulates Transcription of MEK1 and MEK2

SMAD2 phosphorylation/SMAD4 activation is not the main role of MEKK1 (encoded by MAP3K1), which is usually recognised as a MAP3K for the JNK pathway in the context of stress responses, and the MAPK pathway leading to activation of MEK1/2 (MAP2K1/2. MEK1 and MEK2 are dual-specificity kinases that uniquely activate extracellular signal regulated kinases ERK1 and ERK2 by threonine AND tyrosine phosphorylation, and ERKs are their sole known substrates.

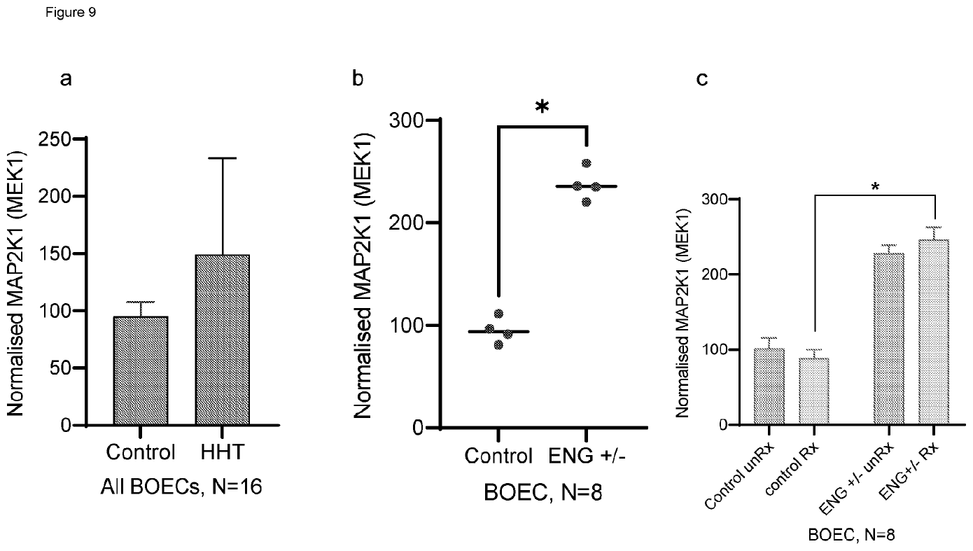

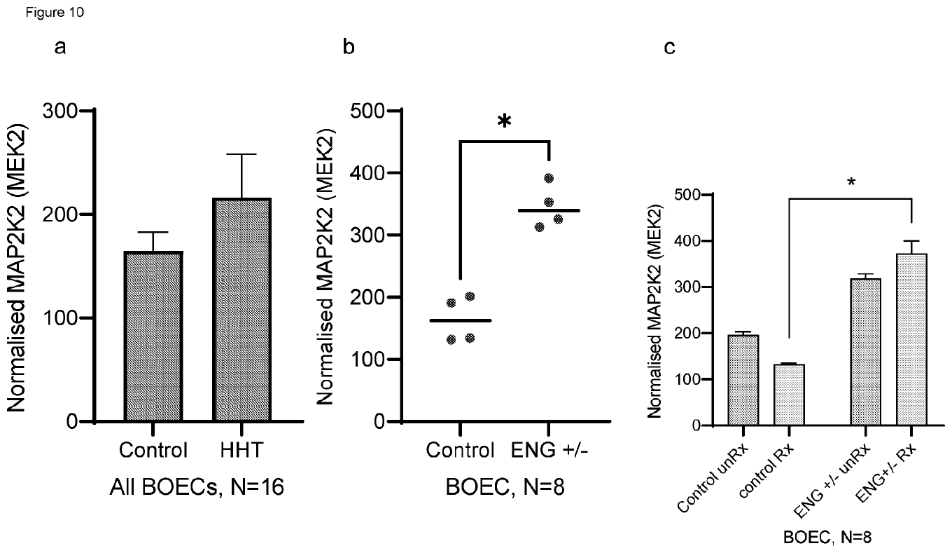

RNAseq was carried out on BOECs from HHT patients and healthy controls to investigate MEK1/MEK2 transcription levels, using RBM45 normalisation to normalise for PTC-related stress. As shown in FIG. 9, MEK1 transcripts were observably increased in BOECs from HHT patients (FIG. 9A), as were MEK2 transcripts (FIG. 10A).

As the available clinical data to-date relates to a patient with an ENG^{+/-} pathogenic variant, the expression of MEK1 and MEK2 was further investigated in HHT BOECs with an ENG^{+/-} pathogenic variant. MEK1 transcripts were observably increased in ENG^{+/-} BOECs (FIG. 9B), as were MEK2 transcripts (FIG. 10B). Significantly, treatment with BMP9 had no effect on MEK1/2 transcript levels (FIGS. 9C and 10C).

Taken together, the data provide evidence that ENG^{+/-} HHT BOECs increase transcripts for MEK1 and MEK2. These gatekeepers for ERK1/2 phosphorylation, and in the setting of already increased, and particularly dynamic stress-increased MAP3K1 activity, are predictive of exuberant phosphorylation of ERK1/ERK2, which can in turn cause result in HHT-associated telangiectasia, HHT-associated haemorrhage and HHT-associated anaemia.

### Example 7—BOECs from HHT Patients have Increased PTC Transcripts Compared with Healthy BOECs

To investigate the level of stress in BOECs from HHT patients compared with controls, all genes with Gini Coefficients (GCs) of less than 0.15 were identified, and these were aligned across all BOEC data sets. The 5 genes with the lowest reported GCs in all data—NXF1, RBM45, HNRNPK (Heterogeneous Nuclear Ribonucleoprotein K), COX4/1 (Cytochrome c oxidase subunit 4 isoform 1) and SF3B2 (Splicing Factor 3b Subunit 2) had been in wet laboratory experiments, and their rankings were displayed separately for clarity.

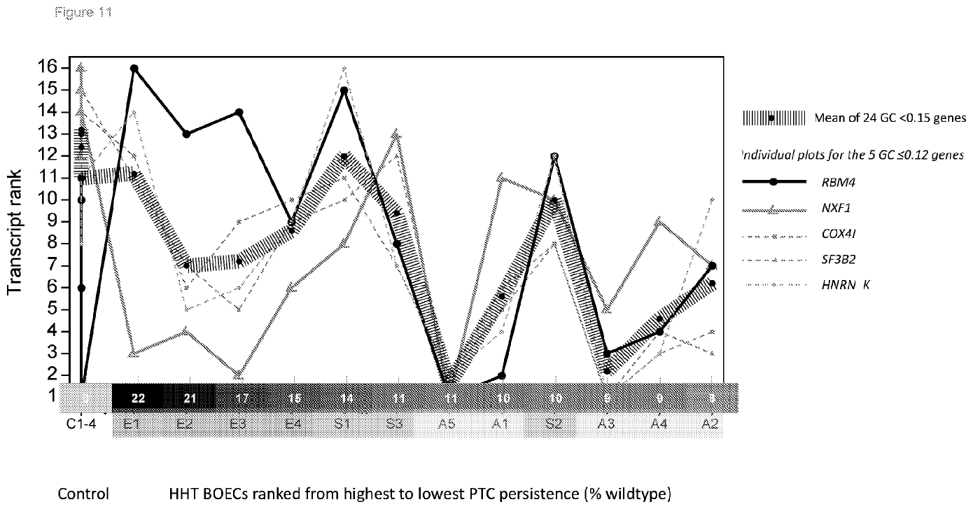

The HHT BOECs were ranked according to their PTC persistence (which is a measure of cellular stress, with PTC persistence increasing as stress increases), and the transcript levels of these 5 genes plotted against PTC persistence (FIG. 11). As shown in FIG. 11, for HNRNPK, COX411 and SF3B2 there is similar variation in the levels of these transcripts across the HHT BOECs, regardless of PTC persistence. However, the level of NXF1 transcript was reduced in the HHT BOECs with the highest levels of PTC persistence, indicating that NXF1 is reduced in conditions of stress. Conversely, the level of RBM45 transcript was increased in the HHT BOECs with the highest levels of PTC persistence, indicating that RBM45 is increased in conditions of stress.

### Discussion

The inventor's own evaluation of over 1,500 HHT patients has demonstrated that HHT-associated telangiectasia which bleed are dynamic, with individual lesions growing and regressing, whereas pulmonary AVMs show no regression and either remain stable or slowly enlarge in adult life.

Whereas some evidence suggests that interventions (e.g. dietary modifications) can have an effect on bleeding, in reviewing more than 900 pulmonary AVM patients, the inventor has observed only one case of spontaneous regression of a pulmonary AVM attributable to an intercurrent pulmonary embolus. Further, clinical experience in the field provides no evidence of any treatment that medically helps pulmonary AVMs. Rather, regression of pulmonary AVMs requires therapeutic embolization obliteration of all feeding arteries to a PAVM sac, or surgical removal, otherwise the sac derives new feeding arteries, and these are from both the pulmonary and the systemic circulation with significant risks of bleeding on acquiring systemic feeders.

The data herein support the conclusion that upregulation of MAP3K1 leads to upregulation of MEK1/MEK2, and that this results in improvement in HHT-associated telangiectasia-associated haemorrhage and improvement in HHT-associated anaemia. HHT mutations do not constitutively activate the MEK1/MEK2 pathways, however the data herein suggest the pathways are impacted secondary to cellular compensations, whereby MEKK-1 (which is a RAF-independent MAP3 kinase (MAP3K) that phosphorylates SMAD4 independently to TGF-B) exhibits increased basal cellular transcript levels. This should not constitutively activate MAPK pathways or upregulate MEKK1-MEK1/2 signalling. However, MAP3K1 can be further upregulated during stress/injury responses (including those involving reactive oxygen species), which supports the inventor's clinical observations that HHT-associated telangiectasia, HHT-associated haemorrhage and HHT-associated anaemia are dynamic, as stress may lead to transient increases in MAP3K1 signalling, and hence transient increases in MEK1/MEK2, exacerbating HHT-associated telangiectasia, HHT-associated haemorrhage and HHT-associated anaemia. Particularly, since MAP3K1 can be upregulated by reactive oxygen species (ROS) stress/injury responses, this supports the inventor's clinical observations that HHT-associated haemorrhage can be precipitated by iron which induces ROS, and that endothelial cells (the cells which line telangiectasia) increase in the blood stream after iron treatment, and enter senescence or undergo non apoptotic cell death in culture after iron treatment, as ROS lead to transient increases in MAP3K1 signalling, and hence transient increases in MEK1/MEK2, exacerbating endothelial injury. Thus, exuberant signalling through the trametinib targets would be greater if cellular stress stimuli cascaded through MEKK1, and lower if any concurrent mitogenic signals (e.g. from angiogenic VEGF) were limited. These considerations appear highly relevant to the development of dynamic telangiectasia in HHT, as exemplified herein, distinct from established AVMs. Indeed, in the patient study presented herein, there was no discernible effect on pre-existing pulmonary AVMs.

The available evidence therefore supports the therapeutic potential of MEK1/MEK2 inhibitors in the treatment of HHT-associated telangiectasia, HHT-associated haemorrhage and HHT-associated anaemia, distinct from AVMs. In particular, trametinib offers additional benefits as a potential therapeutic for treating HHT, as it can be delivered in a well-tolerated oral formulation better suited to regular lifestyles than requirements for intravenous medications.

1-14. (canceled)

15. A method for treating telangiectasia associated with hereditary haemorrhagic telangiectasia (HHT), the method comprising administrating a therapeutically effective amount of a MEK1/2 inhibitor to a patient in need thereof.

16. The method according to claim 15, wherein said inhibitor treats or prevents HHT induced anaemia and/or haemorrhage.

17. The method according to claim 15, wherein said inhibitor is trametinib.

18. (canceled)

19. The method according to claim 15, wherein the telangiectasia is nasal telangiectasia.

20. The method according to claim 16, wherein the HHT induced anaemia is associated with recurrent haemorrhages and/or the HHT induced haemorrhage is recurrent.

21. The method according to claim 16, wherein the anaemia and/or haemorrhage is not responsive to dietary iron supplements.

22. The method according to claim 16, wherein the anaemia and/or haemorrhage would otherwise require treatment comprising surgery, an anti-angiogenic agent and/or blood transfusion.

23. The method according to claim 17, wherein trametinib is administered less than 2 mg/day.

24. The method according to claim 15, wherein the administering comprises oral, intravenous, or subcutaneous administration.

25. The method according to claim 24, wherein the administering comprises oral administration of trametinib.

26. The method according to claim 15, wherein the administering is at a daily, weekly, fortnightly, or monthly interval.

27. The method according to claim 26, wherein the administering is at a daily interval.

28. A method of identifying a patient as suitable for use of a MEK1/2 inhibitor for treating or preventing telangiectasia associated with HHT, said method comprising determining and/or quantifying one or more of a patient's:
(a) red cell indices;
(b) impaired activity;
(c) lack responsiveness to dietary or oral iron supplements; and/or
(d) lack of tolerance to bleeding and/or anaemia.


SEQUENCE LISTING (4 sequences)

SEQ ID NO: 1 - DNA, 2547 bp, Homo sapiens
  source 1..2547: mol_type = other DNA
agtccctcccagggccgcttcgcagagcggctaggagcacggcggcggcggcactttccccggcaggagctggagctgggctctggtgcgcgcgcggctgtgccgcccgagccggagggactggttggttgagagagagagaggaagggaatcccgggctgccgaaccgcacgttcagcccgctccgctcctgcagggcagcctttcggctctctgcgcgcgaagccgagtcccgggcgggtggggcgggggtccactgagaccgctaccggcccctcggcgctgacgggaccgcgcggggcgcacccgctgaaggcagccccggggcccgcggcccggacttggtcctgcgcagcgggcgcggggcagcgcagcgggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaattttggtgaatgtgggtagtcattcttacaattgcactgctgttcctgctccatgactggctgtctgcctgtattttcgggattctttgacatttggtggtactttattcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattattttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggatgaaagcta

SEQ ID NO: 2 - protein, 393 aa, Homo sapiens [sequence omitted]

SEQ ID NO: 3 - DNA, 1727 bp, Homo sapiens
  source 1..1727: mol_type = other DNA
ctctcggactcgggctgcggcgtcagccttcttcgggcctcggcagcggtagcggctcgctcgcctcagccccagcgcccctcggctaccctcggcccaggcccgcagcgccgcccgccctcggccgccccgacgccggcctgggccgcggccgcagccccgggctcgcgtaggcgccgaccgctcccggcccgccccctatgggccccggctagaggcgccgccgccgccggcccgcggagccccgatgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtgacagtggccgggctccctgcgtcccgctggtgacctgcccaccgtccctgtccatgccccgcccttccagctgaggacaggctggcgcctccacccaccctcctgcctcacccctgcggagagcaccgtggcggggcgacagcgcatgcaggaacgggggtctcctctcctgcccgtcctggccggggtgcctctggggacgggcgacgctgctgtgtgtggtctcagaggctctgcttccttaggttacaaaacaaaacagggagagaaaaagcaaa

SEQ ID NO: 4 - protein, 400 aa, Homo sapiens [sequence omitted]